(19) United States
(12) Patent Application Publication
(10) Pub. No.: US 2019/0227063 A1
(43) Pub. Date: Jul. 25, 2019

(54) METHODS AND COMPOSITIONS FOR T-CELL EPITOPE SCREENING

(71) Applicant: MEMORIAL SLOAN KETTERING CANCER CENTER, New York, NY (US)

(72) Inventors: David A Scheinberg, New York, NY (US); Ron Gejman, New York, NY (US)

(21) Appl. No.: 16/333,109

(22) PCT Filed: Sep. 16, 2017
(86) PCT No.: PCT/US17/51938

§ 371 Date: Mar. 13, 2019

Related U.S. Application Data
(60) Provisional application No. 62/395,577, filed on Sep. 16, 2016.

(51) Int. Cl. G01N 33/569; C12N 15/86; C40B 30/06; C12N 5/0783

(52) U.S. Cl. CPC G01N 33/56977; C12N 15/86; C12N 2015/8518; C12N 5/0636; C12N 2740/13041; C40B 30/06

(57) ABSTRACT

The present invention provides new and improved methods for screening for and/or identifying T cell epitopes, as well as various assays and compositions (such as nucleic acid molecules, vectors, viruses, peptides, libraries, and cells), that are useful in carrying out such methods. Such methods and compositions can be used to predict and/or study the toxicity and off-target effects of T-cells, TCRs, or TCR-like molecules.

### CROSS-REFERENCE TO RELATED APPLICATIONS

This application claims the benefit of priority of U.S. Provisional Patent Application No. 62/395,577 filed on Sep. 16, 2016, the contents of which are hereby incorporated by reference in their entirety.

### STATEMENT OF GOVERNMENT SUPPORT

This invention was made with government support under grant numbers CA055349 and CA200327 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Sep. 15, 2017, is named MSKCC_018_WO1_SL.txt and is 29,629 bytes in size.

### INCORPORATION BY REFERENCE

For the purposes of only those jurisdictions that permit incorporation by reference, all of the references cited in this disclosure are hereby incorporated by reference in their entireties (numbers in parentheses or superscript following text in this patent disclosure refer to the numbered references provided in the “Reference List” section of this patent specification). In addition, any manufacturers' instructions or catalogues for any products cited or mentioned herein are incorporated by reference. Documents incorporated by reference into this text, or any teachings therein, can be used in the practice of the present invention.

### BACKGROUND

T cells express T cell receptors or “TCRs” that bind to 8-11 amino acid peptides “presented” on the cell surface in complex with Major Histocompatibility Complex (“MHC”) molecules. (In humans, MHC molecules are also known as Human Leukocyte Antigen (“HLA”) molecules). MHC molecules are expressed on the surface of all nucleated human cells. The peptides presented on MHC molecules can be derived from both intracellular and extracellular proteins. Thus, unlike antibodies—which bind to extracellular proteins—T cells (including engineered T cells), TCRs, and “TCR-like” molecules can bind to, and can be used to target, previously un-targetable intracellular proteins, such as intracellular oncogene products. Engineered T cells include “Chimeric Antigen Receptor T Cells” (“CAR-T cells”). Man-made “TCR-like” molecule formats include soluble TCRs, TCR mimic antibodies (TCRm) and their various forms¹, Immune Mobilizing Monoclonal TCRs Against Cancer (“ImmTACs”), and Bi-Specific T Cell Engagers (“BITES”).

Therapeutic drugs designed to activate, block, or mimic the functions of the immune system are some of the most promising new modalities for the treatment of cancer. For example, one promising class of cancer immunotherapies involves engineering T cells, TCRs or “TCR-like” molecules to specifically target cancer cells for destruction. Another promising methodology is immune checkpoint blockade (“ICB”), which re-activates the immune system in cancer patients and which is revolutionizing therapy. ICB is thought to work by re-activating T cells that have been turned off by cancer cells. However, the agents that are used to achieve ICB, as well as those used in other cancer immunotherapies, can lead to episodes of serious toxicity, due, for example, to activation of T cells having TCRs that are cross-reactive with both tumor tissue and healthy tissue, or to the administration/use of T cells, TCRs or TCR-like molecules that are cross-reactive with both tumor tissue and healthy tissue. For example, a recent clinical trial of an affinity-enhanced TCR against the MAGE-A3 protein was ended after two patients died from cardiogenic shock shortly after infusion of the TCR², and it was discovered that the TCR was cross-reactive with an epitope encoded by the Titin protein.³

T cells and TCRs also play an important role in other disease areas. For example, in patients with infectious diseases, cells of the immune system, by use of their TCRs, recognize epitopes on infected cells that are presented on MHC molecules and mark them for destruction. And in certain autoimmune diseases, a patient's TCRs may recognize and bind to MHC-presented peptides from normal cells—and thereby mark the patients' normal cells for destruction by T cells.

In view of all of the above, there is a need in the art for efficient methods of identifying the target epitopes of TCRs (such as those expressed by the T cells engendered by ICB, and those targeted by, or mimicked by, cancer immuno-therapeutics, anti-infective agents, and/or anti-autoimmunity agents), as well as for identifying “off-target” epitopes that are cross-reactive with such TCRs (or TCR-like molecules)—so that therapeutics can be developed that are not only highly specific but that also do not target normal healthy tissue.

There is currently a dearth of such methods. This is due, in part, to the difficulty in producing libraries of peptides that represent the full-range of possible TCR epitopes (both true target epitopes and off-target epitopes). For example, while there are approximately 20 million, 9-10 amino acid long peptides that can be encoded by the human genome, fewer than 5% (˜700,000) of these peptides are predicted in silico to bind to a given type of HLA molecule. Also, there are an enormous number of potential peptide ligands of MHC molecules that could potentially cross-react with TCRs and therapeutic TCR-like molecules. For example, the most prevalent human MHC allele, HLA-A*02:01, binds to peptides with hydrophobic residues in the 2^{nd} and last position of the peptide. To date the process of identifying cross-reactive targets of TCRs or TCR-like molecules has proved to be very challenging—even when crystal structure information is available—in part because peptides can bind to MHCs in non-canonical manners.⁷ Traditionally, the identification of cross-reactive epitopes of TCRs and TCR-like molecules has been a long, iterative process, where what is learned in one round of testing informs the next targets to be tested.

While some methods for screening TCRs and TCR-like molecules against peptide ligands have been described in recent years,^{4, 5} to date such methods have met with limited success. For example, Birnbaum and colleagues recently developed such a peptide-MHC (“pMHC”) yeast display library of ˜2.1×10⁸ antigen minigenes.⁵ Using Birnbaum's system, cells that bound to soluble TCRs were purified with magnetic beads and then subject to high throughput sequencing.⁵ After four rounds of selection, Birnbaum and colleagues identified hundreds of peptides that were cross-reactive with five distinct mouse TCRs.⁵ However, the authors failed to identify the original epitopes to which the TCR was known to bind.⁵ This was not surprising because—given the enormous possible sequence diversity in 10 amino acid peptides (˜10¹³ peptides)—it was unlikely that any given peptide would have been found in their library.

Furthermore, previously-developed methods for screening TCRs and TCR-like molecules suffer from several other disadvantages. For example, such prior methods typically: (1) involve yeast or insect cell display technologies—such that the generated peptide-MHC complexes may not be representative of those presented by human cells, (2) involve displaying peptides that are covalently linked to MHC molecules using a flexible linker—which can affect the structure and shape of the antigen near the linker site and therefore will alter TCR binding, (3) generate peptide diversity using random cloning approaches—yielding libraries that include many irrelevant peptides (i.e. those not found in the human genome) and that may not include all relevant peptides, (4) have to be reconstructed each time a different MHC allele is to be used for peptide display.

### SUMMARY OF THE INVENTION

The present invention addresses the various needs in the art described above.

In one aspect the present invention provides antigen presentation and TCR binding/screening methods that have the following advantages over prior systems: (1) They can utilize mammalian cells; (2) They do not require covalent linkage of MHC molecules to the peptides displayed on the MHC molecules; (3) They can allow precisely defined HLA-presentable antigens to be expressed; (4) They can be tailored to express peptide antigens that are most likely to bind to or be cross-reactive with TCRs or TCR like molecules; (5) They are single-copy competent methods, and can therefore be used for pooled library screens of large numbers (tens of thousands) of different peptides/TCR epitopes; (6) The vectors used do not have to be re-engineered every time a different MHC molecule is to be used for peptide display because the methods can utilize MHC molecules expressed by the cells in which the assays are performed (to test different MHCs the same vectors can simply be delivered to cells expressing different MHC molecules); and (7) The antigen is expressed in the MHC in exactly the same structure (shape) as an actual antigen for recognition and therefore should precisely mimic the epitope seen by the TCR-based molecules. The details of such methods are described more fully in the Detailed Description and Examples sections of this patent disclosure.

In another aspect, the present invention provides various assays that can be used to carry out such antigen presentation and TCR binding/screening methods. In some embodiments, such assays utilize cells that are deficient in the Transporter Associated with Antigen Processing ½ or “TAP½” proteins—which normally deliver cytosolic peptides into the endoplasmic reticulum (ER), where they bind to nascent MHC class I molecules. These TAP½ deficient cells have very low levels of endogenous antigen presentation, despite having high levels of MHC-I expression. Exemplary TAP½ deficient cell types include, but are not limited to, T2 cells.

In yet another aspect the present invention provides novel nucleic acid molecules (and vectors and/or viruses comprising such nucleic acid molecules), that can be used to carry out such antigen presentation and TCR binding/screening methods, and that, when introduced into cells, allow the delivery of defined peptides directly into the endoplasmic reticulum (ER) of cells, where they can form peptide-MHC (“pMHC”) complexes.

In yet another aspect the present invention provides libraries of such nucleic acid molecules. The libraries provided by the present invention can be either “focused” libraries or “random” libraries—depending on their intended use. For example, if the library is to be used to identify epitopes that cross-react with a known T-cell, TCR or TCR-like molecule, a focused library can be generated and used to maximize the chance of finding cross-reactive epitopes. However, in embodiments where there is no prior knowledge of the epitopes that might be identified, a random library (i.e. a library containing randomly generated or randomly selected peptides) may be preferable.

These and other embodiments of the invention are further described in the “Brief Description of the Figures,” “Detailed Description,” “Examples,” “Claims,” “Figures,” and “Sequence Listing” sections of this patent disclosure. Furthermore, one of skill in the art will recognize that the various embodiments of the present invention described can be combined in various different ways, and that such combinations are within the scope of the present invention.

### BRIEF DESCRIPTION OF THE FIGURES

FIG. 1 A-C: Overview of exemplary embodiment of the PresentER Retroviral System. (A) In this example, the PresentER system is based on an MSCV retroviral vector. The peptide antigen minigene is driven by the MSCV LTR and encodes an endoplasmic reticulum (ER) targeting sequencing followed by the precise peptide to be expressed, followed by a stop codon. The vector contains a puromycin resistance gene and GFP driven by PGK. (B) An exemplary PresentER construct—having a leader sequence from MMTV gp70 protein (SEQ ID NO: 2). (C) An overview of how the virus is created and used to generate infected T2 cells.

FIG. 2 A-D: The PresentER system can encode MHC-bound and TCR-recognizable ligands. T2 cells were spinoculated with retrovirus encoding 5 different MHC ligands: Single, live (DAPI negative), GFP-positive cells were gated and ESK1 or Pr20 binding levels were assessed. (A) Flow cytometry histograms showing that only cells expressing RMF bind ESK1 at levels greater than ˜1,000 fluorescence units (FU). (B) Quantification of the frequency of PresentER cells with FU greater than the threshold. (C) Flow cytometry histograms showing that only cells expressing ALY bind Pr20 at levels greater than ˜1,100 fluorescence units (FU). (D) Quantification of the frequency of PresentER cells with FU greater than the threshold.

FIG. 3: An ER targeting sequence is essential for PresentER antigen presentation. T2 cells were spinoculated with a PresentER minigene encoding RMF or ALY. T2s were also spinoculated with minigenes encoding one of two scrambled ER sequences, followed by RMF or ALY. Only the correct ER targeting sequences promoted ESK1 and Pr20 binding to cells encoding their cognate antigen.

FIG. 4 A-D: The PresentER system can be used to activate T cells and present epitopes to soluble T cell receptors (TCR). A soluble, fluorescently labeled anti-NLV TCR multimer from Altor Biosciences was used and T2 cells expressing RMF, ALY or NLV PresentER minigenes were stained. (A) The TCR bound specifically to T2s expressing the CMVpp65 antigen. (B) Quantification of soluble anti-NLV TCR binding to PresentER T2s. (C) A soluble, fluorescently labeled anti-“LLF” peptide (i.e. LLFGYPVYV—SEQ ID NO. 17) TCR tetramer was made from the A6 T cell receptor (17, Utz et al 1996) and T2 cells expressing “RMF” peptide, “ALY” peptide or “LLF” peptide PresentER minigenes were stained. Amino acid sequences of the “RMF,” “ALY,” and “LLF” peptides are SEQ ID Nos. 36, 37 and 17, respectively. Nucleotide sequences of PresentER minigenes comprising sequences that encode the “RMF,” “ALY,” and “LLF” peptides are SEQ ID Nos. 11, 12 and 18, respectively. (D) An IFNγ release assay was performed on T2 cells spinoculated with RMF, ALY or NLV encoding PresentER minigenes or pulsed overnight with RMF, ALY or NLV peptide at 20 μg/ml. IFNγ was specifically released by anti-NLV T cells only when co-cultured with T2s expressing PresentER-NLV or pulsed with NLV peptide. Amino acid sequences of the “RMF,” “ALY,” and “NLV” peptides are SEQ ID Nos. 36, 37 and 47, respectively.

FIG. 5 A-D: The PresentER vector is a single-copy competent vector. T2 cells were spinoculated with serial dilutions of PresentER-RMF and PresentER-ALY virus. Cells were spinoculated with 1 ml, 200 μl, 100 μl and 20 μl of virus per 250 k cells in duplicate. They were co-stained with ESK1 and Pr20 and the percent Pr20 and ESK1 binding was evaluated by flow cytometry as function of (A-B) volume of virus (titer) or (C-D) percent of cells infected (functional titer).

FIG. 6 A-D: Schematic of exemplary PresentER minigene cloning and amplification for high throughput sequencing. (A) This exemplary PresentER minigene precursor consists of the ER signal sequence followed by a removable ˜200 nt cassette bounded by SfiI restriction sites. The removable cassette, while not essential, provides a technical aid to visualize restriction enzyme digestion when using this precursor to generate the final PresentER minigene vectors. This exemplary vector has built in SP1, SP2 and SP3 binding sites for Illumina sequencing. SEQ ID NO. 9 is a representative PresentER minigene precursor sequence. (B) The antigen portion of the minigene can be synthesized as a ˜75 nt oligonucleotide bounded by SfiI sites and primer binding sites to allow amplification of the oligo before digestion and cloning. Cloning is performed by digesting both the vector backbone and the antigen with SfiI and ligating the two pieces together with T4 ligase. (C) The DNA context of the fully cloned PresentER minigene with P5 and P7 primer amplification sites shown. (D) The amplicons formed by P5/P7 primer amplification with SP1, SP2, SP3, antigen and index all displayed. There are two read 1 sequencing primer hybridization sites: SP1 (for standard Illumina sequencing) and CustomPrimer33 (SEQ ID NO. 34), which overlies the entire constant region and begins sequencing at the first nucleotide of the variable antigen region, and can thus be used for sequencing of any PresentER inserts.

FIG. 7 A-D: PresentER library validation sequencing and screening for ESK1 cross-reactive targets. (A) A PresentER library of ESK1 and Pr20 cross-reactive epitopes was amplified with P5 and P7 primers and submitted for Illumina sequencing to determine if all minigenes were well represented. A histogram showing the abundance of each minigene in the library shows that the library is normally distributed and well represented. (B) The PresentER library was screened for ESK1 binding epitopes and the results plotted by netMHCPan HLA-A*02:01 affinity to HLA IC₅₀ versus enrichment for ESK1 binding. Previously known ESK1 ligands^{8,10} are marked as triangles and previously known ESK1 non-binders are marked by diamonds. Peptides that were enriched for ESK1 binding in the PresentER screen and were subsequently validated by peptide pulsing are marked with squares. Peptides which were enriched in the screen but did not validate by peptide pulsing are marked by the “+” symbol. Many peptides are depleted, some are enriched and most are unchanged. (C) Boxplots of HLA-A*02:01 affinity for the whole library, ≥5×ESK1 depleted minigenes and ≥5×ESK1 enriched minigenes. (D) The peptides selected for inclusion in the Pr20 genomic off-target library and single amino acid mismatches to ALYVDSLFFL (SEQ ID NO: 37), plotted by predicted HLA A*02:01 affinity and enrichment for ESK1 binding. The symbols are defined in the legend to FIG. 7B.

FIG. 8: An exemplary PresentER library was screened for ESK1 binding epitopes and the results plotted by netMHCPan HLA-A*02:01 affinity to HLA IC₅₀ versus enrichment for ESK1 binding. The symbols are defined in the legend to FIG. 7B. In this figure only the ESK1 genomic off-target epitopes and single-amino acid mismatch to RMF are plotted.

FIG. 9 An exemplary PresentER library was screened for Pr20 binding epitopes and the results plotted by netMHCPan HLA-A*02:01 affinity to HLA IC₅₀ versus enrichment for Pr20 binding. The symbols are defined in the legend to FIG. 7B.

### DETAILED DESCRIPTION

The present invention provides new and improved methods for screening for and/or identifying T cell epitopes, as well as various assays and compositions (such as nucleic acid molecules, vectors, viruses, peptides, libraries, and cells), that are useful in carrying out such methods. Such methods and compositions have a variety of uses. For example, such methods and compositions can be used to predict and/or study the toxicity and/or off-target effects of TCR-based drugs or of T-cells, TCRs, or TCR-like molecules.

Some of the main embodiments of the present invention are described in the Summary of the Invention, Examples, Claims, and Figures sections of this patent disclosure. This Detailed Description section provides certain additional description, and is intended to be read in conjunction with all other sections of the present patent disclosure. Furthermore, the sub-headings provided below, and throughout this patent disclosure, are provided for convenience only and are not intended to denote limitations of the various aspects or embodiments of the invention, which are to be understood by reference to the specification as-a-whole.

### Definitions & Abbreviations

As used in this patent disclosure, including the appended Claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Units, prefixes, and symbols are denoted in their Systeme International de Unites (SI) accepted form. Numeric ranges provided herein are inclusive of the numbers defining the range.

Where a numeric term is preceded by “about” or “approximately,” the term includes the stated number and values ±10% of the stated number.

The terms “T cells,” “T cell receptors” (“TCRs”), and “TCR-like molecules” are used in accordance with their usual meaning in the art. “TCR-like molecules” include, but are not limited to, soluble TCRs, TCR mimic antibodies (TCRm) and their various forms′, Immune Mobilizing Monoclonal TCRs Against Cancer (“ImmTACs”), and Bi-Specific T Cell Engagers (“BITES”).

As used herein, the term/abbreviation “ALY” refers to the amino acid sequence ALYVDSLFFL (SEQ ID NO. 37) or a peptide having that amino acid sequence. In some instances, as will be clear from the context in which the term/abbreviation is used, such term/abbreviation may refer to a nucleotide sequence that encodes such an amino acid sequence or peptide.

As used herein, the term/abbreviation “EW” refers to the amino acid sequence QLQNPSYDK (SEQ ID NO. 42) or a peptide having that amino acid sequence. In some instances, as will be clear from the context in which the term/abbreviation is used, such term/abbreviation may refer to a nucleotide sequence that encodes such an amino acid sequence or peptide.

As used herein, the term/abbreviation “Flu” refers to the amino acid sequence GILGFVFTL (SEQ ID NO. 43) or a peptide having that amino acid sequence. In some instances, as will be clear from the context in which the term/abbreviation is used, such term/abbreviation may refer to a nucleotide sequence that encodes such an amino acid sequence or peptide.

As used herein, the term/abbreviation “LLF” (or “HTLV1 Tax”) refers to the amino acid sequence LLFGYPVYV (SEQ ID NO. 17) or a peptide having that amino acid sequence. In some instances, as will be clear from the context in which the term/abbreviation is used, such term/abbreviation may refer to a nucleotide sequence that encodes such an amino acid sequence or peptide.

As used herein, the term/abbreviation “RMF” refers to the amino acid sequence RMFPNAPYL (SEQ ID NO. 36) or a peptide having that amino acid sequence. In some instances, as will be clear from the context in which the term/abbreviation is used, such term/abbreviation may refer to a nucleotide sequence that encodes such an amino acid sequence or peptide.

As used herein, the term/abbreviation “WT1 239” refers to the amino acid sequence NQIVINLGATL (SEQ ID NO. 44) or a peptide having that amino acid sequence. In some instances, as will be clear from the context in which the term/abbreviation is used, such term/abbreviation may refer to a nucleotide sequence that encodes such an amino acid sequence or peptide.′

Other terms are defined elsewhere in this patent specification, or else are used in accordance with their usual meaning in the art.

Methods for Screening for and/or Identifying T Cell Epitopes

In some embodiments, the present invention provides various methods for screening for and/or identifying T cell epitopes. In some embodiments, such methods involve contacting an “engineered target cell” with a T cell, a TCR, or a TCR-like molecule, and performing an assay to determine whether the T cell, TCR, or a TCR-like molecule binds to the engineered target cell, and/or to measure the strength of any such binding. The “engineered target cell” contains a recombinant PresentER nucleic acid molecule—as further described below. Expression of the PresentER nucleic acid molecule in the engineered target cell results in the cell displaying the peptide encoded by the PresentER nucleic acid molecule on its cell surface in association (e.g. non-covalent association) with an MHC molecule as a peptide-MHC (pMHC) complex. In some embodiments the peptide is not covalently attached to the MHC molecule. In some embodiments, the “engineered target cell” is produced using one of the methods described herein. In some embodiments, the “engineered target cell” comprises a nucleic acid molecule, vector, virus, peptide, or engineered peptide-MHC (pMHC) complex as described herein.

In some such methods, the method is a library screening method—comprising contacting a population of engineered target cells with T cells, TCRs, or TCR-like molecules, and performing an assay to determine whether any of the T cells, TCRs, or TCR-like molecules bind to any of the engineered target cells in the population of engineered target cells, and/or to measure the strength of any such binding. In such library screening methods, the population of engineered target cells comprises a library of nucleic acid molecules (as further described elsewhere herein) and different cells in the population of engineered target cells express different library nucleic acid molecules and express/display different engineered peptide-MHC (pMHC) complexes on their cell surface.

In some embodiments, when performing such screening methods and/or library screening methods, the step of contacting the engineered target cells with T cells, TCRs, or TCR-like molecules is performed in vitro. In some embodiments, when performing such screening methods and/or library screening methods the step of contacting the engineered target cells with T cells, TCRs, or TCR-like molecules is performed in vivo, such as, for example in a suitable animal model. Similarly, in some embodiments, when performing such screening methods and/or library screening methods, the step of performing an assay to determine whether any of the T cells, TCRs, or TCR-like molecules bind to any of the engineered target cells is performed in vitro, while in other embodiments, the step of performing an assay to determine whether any of the T cells, TCRs, or TCR-like molecules bind to any of the engineered target cells is performed in vivo, such as, for example in a suitable animal model.

Assays to determine whether any of the T cells, TCRs, or TCR-like molecules bind to any of the engineered target cells can be performed using any suitable methods known in the art. For example, in some of those embodiments where the assay is performed in vitro, the assay may comprise detecting and/or measuring binding of the T cells, TCRs, or TCR-like molecules bind to the engineered target cells by performing flow cytometry, fluorescence activated cell sorting (FACS) by using an affinity column, by using another solid-phase affinity system, or based on measuring some signal associated with binding of T cells, TCRs, or TCR-like molecules to the engineered target cells—including, but not limited to, IFN gamma secretion. Similarly, in some of those embodiments where the assay is performed in vivo, the assay may comprise detecting and/or measuring binding of the T cells, TCRs, or TCR-like molecules bind to the engineered target cells based on detecting and/or measuring some signal associated with binding of T cells, TCRs, or TCR-like molecules to the engineered target cells, such as an immune response, or an indicator of an immune response.

In some embodiments the methods for screening for and/or identifying T cell epitopes described above and/or elsewhere herein further comprise separating engineered target cells that bind to the T cells, TCRs, or TCR-like molecules from those that don't bind the T cells, TCRs, or TCR-like molecules, and/or separating engineered target cells that bind to the T cells, TCRs, or TCR-like molecules with high (or higher) affinity from those that bind the T cells, TCRs, or TCR-like molecules with low (or lower) affinity. In such embodiments, the step of “separating” the different categories of engineered target cells can be performed using any suitable method for cell separation known in the art. For example, in some embodiments, the separation step is performed using FACS. Similarly, in some embodiments, the separation step is performed using magnetic bead sorting.

In some embodiments, the methods for screening for and/or identifying T cells, TCRs, or TCR-like molecules described above and/or elsewhere herein further comprise isolating and/or amplifying a nucleic acid molecule encoding the peptide component of the pMHC complex expressed/displayed by the engineered target cell.

In some embodiments, the methods for screening for and/or identifying T cell epitopes described above and/or elsewhere herein further further comprise sequencing the nucleic acid molecule encoding the peptide component of the pMHC complex expressed/displayed by the engineered target cell.

The methods for screening for and/or identifying T cell epitopes described above and/or elsewhere herein can be performed using T cells, TCRs, or TCR-like molecules. Where T cells are used, various different types of T cells can be used. In some embodiments, the T cells are naturally occurring T cells. In some embodiments, the T cells are those elicited in human patients in response to Immune Checkpoint Blockade (ICB) therapy. In some embodiments, the T cells are cultured cells from a T cell line. In some embodiments, the T cells are engineered T cells. In some embodiments, the engineered T cells are “Chimeric Antigen Receptor T Cells” (“CAR-T cells”). Where TCRs are used, various different types of TCRs can be used. In some embodiments, the TCRs are naturally occurring TCRs cells. In some embodiments, the TCRs are engineered TCRs. Various different types of TCR-like molecules can also be used in carrying out the methods described above and elsewhere herein. In some embodiments, the TCR-like molecules are selected from the group consisting of: soluble TCRs, TCR mimic antibodies (TCRm), Immune Mobilizing Monoclonal TCRs Against Cancer (“ImmTACs”), and Bi-Specific T Cell Engagers (“BITES”).

### Nucleic Acid Molecules & Peptides

In some embodiments, the present invention provides certain nucleic acid molecules, as well as vectors, libraries, viruses and/or cells that comprise such nucleic acid molecules, and various methods that involve the use of such nucleic acid molecules. Such nucleic acid molecules are recombinant nucleic acid molecules—i.e. nucleic acid molecules that are made by man, for example by bringing together nucleic acid sequences from multiple sources, and/or by modifying nucleic acid sequences that are found in nature. As such, the nucleic acid molecules described herein are not naturally occurring. While the nucleic acid molecules described herein may contain nucleic acid sequences that occur in nature (such as, for example, naturally occurring ER signal sequences), the nucleic acid molecules as-a-whole are man-made.

For example, the present invention provides nucleic acid molecules that can be used to express/display a peptide, or a library of peptides, on the surface of a cell (such as an engineered target cell) in association with an MHC molecule. These nucleic acid molecules may be referred to generically herein as “PresentER” nucleic acid molecules. (Similarly, the vectors, libraries, viruses and/or cells that comprise such nucleic acid molecules may be referred to generically herein as “PresentER” vectors, libraries, viruses and/or cells, and the methods of use of such nucleic acid molecules vectors, libraries, viruses and/or cells may be referred to generically herein as PresentER methods. Such nucleic acid molecules (i.e. PresentER” nucleic acid molecules) comprise: (a) a nucleotide sequence that encodes an ER signal sequence, and (b) a nucleotide sequence that encodes a peptide downstream of, and in frame with, the nucleotide sequence that encodes the ER signal sequence. These “PresentER” nucleic acid molecules encode a fusion protein comprising peptide with an N-terminal ER signal sequence. Typically, there will also be a stop codon downstream of the nucleotide sequence that encodes the peptide in the PresentER nucleic acid molecule. Typically, the nucleic acid molecules will be operably linked to a promoter. Any promoter that will allow expression of the peptide/fusion in the desired target cell type can be used. For example, promoters that enable constitutive expression or regulated expression may be used. And promoters that enable tissue-specific or cell-specific expression may be used. In some embodiments, the nucleic acid molecule also comprises a selectable marker. In some embodiments, such nucleic acid molecules also comprise nucleotide sequences upstream and/or downstream of the nucleotide sequence that encodes the peptide that can be used to facilitate the isolation, amplification, and/or sequencing of the nucleotide sequence that encodes the peptide.

In some embodiments, the ER signal sequence used in such nucleic acid molecules may be any suitable ER signal sequence known in the art. For example, in some embodiments, the ER signal sequence may be selected from those listed in the public signal peptide database available at http://www.signalpeptide.de/. In some embodiments, the ER signal sequence is the MMTV gp70 ER targeting sequence. In some embodiments, the nucleotide sequence that encodes the ER signal sequence comprises MMTV1 (SEQ ID NO. 1). In some embodiments, the nucleotide sequence that encodes the ER signal sequence comprises a modified MMTV gp70 ER targeting sequence referred to as MMTV2 (SEQ ID NO. 5). In some embodiments, the nucleotide sequence that encodes the ER signal sequence comprises SEQ ID NO. 10. ER signal sequences contain a signal peptidase (SPase) cleavage site—allowing the signal sequences to be cleaved off leading to release of the peptide from the ER signal sequence.

In some embodiments, the nucleotide sequence that encodes the peptide is present in the human genome. In some embodiments, the nucleotide sequence that encodes the peptide is present in the human exome. In some embodiments, the peptide is a human proteomic peptide. In some embodiments, the peptide is a viral peptide. In some embodiments, the peptide is a microbial peptide. In some embodiments, the peptide does not exist in nature. In some embodiments, the peptide is known to be, or predicted to be, an MHC ligand. In some embodiments, the peptide is an MHC ligand that is unstable in solution. In some embodiments, the peptide is an MHC ligand that cannot be made synthetically. In some embodiments, the peptide is known to be, or predicted to be, an MHC class I ligand. In some embodiments, the peptide is known to be, or predicted to be, an MHC class II ligand. In some embodiments, the peptide binds to an MHC molecule with an IC₅₀ of 1 nM to 500 nM.

The peptide encoded by the nucleic acid molecule should be of a size that allows its expression/display on an MHC molecule and/or that is such that the peptide is, or comprises, an epitope of a T-cell, TCR, or TCR-like molecule. In some embodiments, the encoded peptide is 8-11 amino acids in length. In some embodiments, the encoded peptide is 8-12 amino acids in length, 8-13 amino acids in length, 8-14 amino acids in length, 8-15 amino acids in length, 8-16 amino acids in length, 8-17 amino acids in length, 8-18 amino acids in length, 8-19 amino acids in length, 8-20 amino acids in length, 8-21 amino acids in length, 8-22 amino acids in length, 8-23 amino acids in length, or 8-24 amino acids in length. In some embodiments, the encoded peptide is 8-25 amino acids in length. Similarly, in some embodiments the lower end of such ranges of peptide lengths may be 7 amino acids in length, or 6 amino acids in length, or 5 amino acids in length, or 4 amino acids in length. One of ordinary skill will recognize that peptides that vary in length from those specified herein can also be used, and that such variant peptides having different lengths fall within the scope of the present invention.

In some embodiments, the nucleotide sequence that encodes the ER signal sequence, and the nucleotide sequence that encodes the acid peptide are separated from one another by a spacer, such as a spacer that encodes one or more amino acids. In some such embodiments, the spacer is a cleavable spacer. In some embodiments, the spacer can be the spacer can be cleaved by an ER-associated peptidase. It should be noted that, ER signal sequences themselves generally comprise a signal peptidase (SPase) cleavage site—which can be cleaved by SPases leading to release of the peptide from the ER signal sequence. However, in some embodiments in may be desirable to include an additional cleavable element in a spacer.

As mentioned above, in some embodiments the nucleic acid molecules also comprise nucleotide sequences upstream and/or downstream of the nucleotide sequence that encodes the peptide that can be used to facilitate the isolation, amplification, and/or sequencing of the nucleotide sequence that encodes the peptide. In some embodiments, such sequences comprise amplification primer binding sites. In some embodiments, such sequences comprise sequencing primer binding sites. In some embodiments, such sequences comprise primer binding sites for use in a high-throughput sequencing method. In some embodiments, such sequences comprise primer binding sites that are barcoded for use in a high-throughput sequencing method. In some embodiments, such sequences comprise Illumina signal sequences. In some embodiments, such sequences comprise P5 and/or P7 Illumina amplification primer binding sites. In some embodiments, such sequences comprise SP1, SP2 and/or SP3 Illumina sequencing primer binding sites. In some embodiments, such sequences comprise restriction enzyme cleavage sites. In some embodiments, such sequences comprise a pair of identical restriction enzyme cleavage sites.

As described above, typically, the nucleic acid molecules described herein will be operably linked to a promoter. Any promoter that is sufficient to drive expression of the nucleic acid molecule in the desired engineered target cell can be used.

As described above, in some embodiments the nucleic acid molecules described herein may also comprise a selectable marker. Any suitable selectable marker may be used. In some embodiments, the selectable marker is an antibiotic resistance gene.

In some embodiments, the nucleic acid molecules described herein may also comprise a detectable marker. Any suitable detectable marker may be used. In some embodiments, the detectable marker encodes a fluorescent protein. In some embodiments, the detectable marker encodes a fluorescent protein selected from the group consisting of GFP, RFP, YFP, and CFP.

In some embodiments, the nucleic acid molecules described herein comprise SEQ ID NO. 1. In some embodiments, the nucleic acid molecules described herein comprise SEQ ID NO. 5. In some embodiments, the nucleic acid molecules described herein comprise SEQ ID NO. 9. In some embodiments, the nucleic acid molecules described herein comprise SEQ ID NO. 10. In some embodiments, the nucleic acid molecules described herein comprise SEQ ID NO. 35. In some embodiments, the nucleic acid molecules described herein comprise SEQ ID NO. 40. In some embodiments, the nucleic acid molecules described herein comprise SEQ ID NO. 41. In some embodiments, the nucleic acid molecules described herein may comprise any of the specific nucleotides identified in the Sequence Listing section of this patent disclosure.

The present invention also provides PresentER cloning cassettes into which a nucleotide sequence encoding a peptide, or a library of such nucleotide sequences, can be inserted. Such cloning cassettes may have any of the characteristics described above for PresentER nucleic acid molecules. Typically, such cloning cassettes comprise one or more restriction sites downstream of the nucleotide sequence that encodes the ER signal sequence into which a nucleotide sequence encoding a peptide, or a library of such nucleotide sequences, can be inserted. SEQ ID NO. 9 and SEQ ID NO. 35 provide exemplary PresentER cloning cassettes. The present invention also provides various primers/oligos that may be useful in generating PresentER nucleic acid molecules. SEQ ID NO. 34 is such a primer/oligo.

In addition to the nucleic acid molecules described above that can be used to express/display a peptide, or a library of peptides, in MHC molecules on the surface of a cell (i.e. the PresentER nucleic acid molecules and PresentER cloning cassettes), the present invention provides numerous other nucleic acid sequences. For example, the present invention provides primer and/or oligo sequences, such as those that may be useful in the construction and/or analysis of “PresentER” nucleic acid molecules, as described further in the Examples section of this patent application, including those identified herein using SEQ ID Nos. 19-34, 38-41, 48-49.

The present invention also provides the nucleic acid sequences of numerous exemplary PresentER nucleic acid molecules—encoding various different ER signal-peptide fusion proteins, as described further in the Examples section of this patent application, including those identified herein using SEQ ID Nos 3, 4, 11-16, and 18. In addition, the present invention also provides amino acid sequences of numerous exemplary molecules, including exemplary “PresentER” molecules comprising ER signal-peptide fusion proteins and exemplary peptides that can be used/expressed using the “PresentER” system, as described further in the Examples section of this patent application, including those identified herein using SEQ ID Nos. 2, 6, 17, 36-37, 42-45 and 47.

The full sequences of the specific exemplary nucleotide and amino acid sequences described herein are provided in the Sequence Listing section of this patent disclosure. A summary of what each of these specific exemplary sequences is, or encodes, is provided in Table 1. Further description regarding each sequence and/or its significance or use, is provided in the Examples section of this patent disclosure and/or in the Figures referred to therein.

TABLE 1

| Sequence | |
|---|---|
| Identification No. | Sequence Summary |
| SEQ ID NO: 1 | MMTV1 (DNA sequence of gp70 ER targeting sequence) |
| SEQ ID NO: 2 | MMTV1 (Protein sequence of gp70 ER targeting sequence) |
| SEQ ID NO: 3 | XhoI-MMTV1-″RMF″ peptide-EcoRI (DNA) |
| SEQ ID NO: 4 | XhoI-MMTV1-″ALY″ peptide-EcoRI (DNA) |
| SEQ ID NO: 5 | MMTV2 (DNA sequence of modified gp70 ER targeting sequence) |
| SEQ ID NO: 6 | MMTV2 (Protein sequence of modified gp70 ER targeting sequence) |
| SEQ ID NO: 7 | MMTV2 Scrambled #1 |
| SEQ ID NO: 8 | MMTV2 Scrambled #2 |
| SEQ ID NO: 9 | XhoI-P7-MMTV2 (SfiI)-cassette-SfiI-SP1-P5-EcoRI |
| SEQ ID NO: 10 | PresentER Oligo |
| SEQ ID NO: 11 | ″RMF″ peptide PresentER Oligo |
| SEQ ID NO: 12 | ″ALY″ peptide PresentER Oligo |
| SEQ ID NO: 13 | ″NLV″ peptide PresentER Oligo |
| SEQ ID NO: 14 | ″EW″ peptide PresentER Oligo |
| SEQ ID NO: 5 | ″Flu″ peptide PresentER Oligo |
| SEQ ID NO: 16 | ″WT1 239″peptide PresentER Oligo |
| SEQ ID NO: 17 | ″HTLV1 Tax″ or ″LLF″ peptide |
| SEQ ID NO: 18 | ″HTLV1 Tax″ or ″LLF″ PresentER Oligo |
| SEQ ID NO: 19 | T7_SfiI |
| SEQ ID NO: 20 | T3_SfiI |
| SEQ ID NO: 21 | P7 Primer Index #1 |
| SEQ ID NO: 22 | P7 Primer Index #2 |
| SEQ ID NO: 23 | P7 Primer Index #3 |
| SEQ ID NO: 24 | P7 Primer Index #4 |
| SEQ ID NO: 25 | P7 Primer Index #5 |
| SEQ ID NO: 26 | P7 Primer Index #6 |
| SEQ ID NO: 27 | P7 Primer Index #7 |
| SEQ ID NO: 28 | P7 Primer Index #8 |
| SEQ ID NO: 29 | P7 Primer Index #9 |
| SEQ ID NO: 30 | P7 Primer Index #10 |
| SEQ ID NO: 31 | P7 Primer Index #11 |
| SEQ ID NO: 32 | P7 Primer Index #12 |
| SEQ ID NO: 33 | P5 Primer |
| SEQ ID NO: 34 | MMTVCustomPrimer33 |
| SEQ ID NO: 35 | PresentER (MMTV2) Cassette |
| SEQ ID NO: 36 | ″RMF″ peptide-i.e. WT1 peptide aa126-134 |
| SEQ ID NO: 37 | ″ALY″ peptide-i.e.-PRAME300-309 |
| SEQ ID NO: 38 | Forward Primer |
| SEQ ID NO: 39 | Reverse Primer |
| SEQ ID NO. 40 | DNA sequence for 5′ end of oligos for cloning of peptide encoding |
| | sequences into PresentER vector using the SfiI restriction enzyme. |
| | Oligo contains the SfiI restriction site and part of the MMTV2 signal |
| | sequence. (Sequences encoding custom/library peptides can be |
| | flanked with SEQ ID NO. 40 and SEQ ID NO. 41 for insertion into the |
| | PresentER vector using SfiI restriction sites). |
| SEQ ID NO. 41 | DNA sequence for 3′ end of oligos for cloning of peptide encoding |
| | sequences into PresentER vector using the SfiI restriction enzyme. |
| | Oligo contains a stop codon and the SfiI restriction site. (Sequences |
| | encoding custom/library peptides can be flanked with SEQ ID NO. |
| | 40 and SEQ ID NO. 41 for insertion into the PresentER vector using |
| | SfiI restriction sites). |
| SEQ ID NO: 42 | ″EW″ Negative control peptide |
| SEQ ID NO: 43 | ″Flu″ Negative control peptide |
| SEQ ID NO: 44 | ″WT1 239' Negative control peptide |
| SEQ ID NO: 45 | AVITAG″ peptide sequence |
| SEQ ID NO: 46 | ″AVITAG″ nucleotide sequence |
| SEQ ID NO: 47 | ″NLV″ peptide-i.e. cytomegalovirus (CMV) pp65 aa495-503 |
| SEQ ID NO: 48 | A6b AviTag Forward oligo/primer |
| SEQ ID NO: 49 | A6b AviTag Reverse oligo/primer |
| SEQ ID NO: 50 | gp70 targeting sequence (modified and unmodified)-amino acid |
| | sequence |
| SEQ ID NO: 51 | gp70 targeting sequence (unmodified)-amino acid sequence |
| SEQ ID NO: 52 | gp70 targeting sequence (modified)-amino acid sequence |

In addition to the specific exemplary sequences that are disclosed herein, one of ordinary skill will recognize that variants of such specified sequences can also be used, and that such variants fall within the scope of the present invention. For example, in some embodiments variants of the specific sequences disclosed herein from other species (orthologs) may be used. Similarly, in other embodiments variants that comprise fragments of any of the specific sequences disclosed herein may be used. Likewise, in some embodiments variants of the specific sequences disclosed herein that comprise one or more substitutions, additions, deletions, or other mutations may be used. In some such embodiments, the variant sequences have at least about 40% or 50% or 60% or 65% or 70% or 75% or 80% or 85% or 90% or 95% or 98% or 99% identity with the specific sequences described herein.

For all embodiments where nucleotide sequences that encode a peptide or protein are provided, the corresponding amino acid sequences (i.e. the amino acid sequences encoded by the nucleotide sequences) also form part of the present invention.

### Libraries

As described above, and in the Examples section of this patent disclosure, in some embodiments the present invention provides libraries of the various PresentER nucleic acid molecules described herein. These may be referred to as libraries of “PresentER” nucleic acid molecules or as “PresentER libraries.” In some embodiments, such libraries comprise multiple (i.e. two or more) different nucleic acid molecules and encode multiple (i.e. two or more) different peptides or ER signal sequence-peptide fusions (“peptide fusions”). In some embodiments, such libraries encode at least 100 different peptides or peptide fusions. In some embodiments, such libraries encode at least 500 different peptides or peptide fusions. In some embodiments, such libraries encode at least 1,000 different peptides or peptide fusions. In some embodiments, such libraries encode at least 5,000 different peptides or peptide fusions. In some embodiments, such libraries encode at least 10,000 different peptides or peptide fusions.

In some such embodiments, the nucleic acid molecules in the library are present in a single-copy competent viral vector. In some such embodiments, the nucleic acid molecules in the library comprise a randomly selected group of nucleic acid molecules. In some such embodiments, the nucleic acid molecules in the library encode a randomly selected group of peptides. In some such embodiments, the nucleic acid molecules in the library comprise, or consist of, nucleic acid molecules that encode peptides known or predicted to bind to an MEW molecule. In some such embodiments, the nucleic acid molecules in the library comprise, or consist of, nucleic acid molecules that encode peptides known, or predicted to bind to an MHC Class I molecule. In some such embodiments, the nucleic acid molecules in the library comprise, or consist of, nucleic acid molecules that encode peptides known or predicted to bind to an MHC Class II molecule. In some such embodiments, the nucleic acid molecules in the library comprise, or consist of, nucleic acid molecules that encode peptides known or predicted to bind to an MHC molecule with an IC₅₀ of 1 nM to 500 nM.

In some such embodiments, the nucleic acid molecules in the library comprise, or consist of, nucleic acid molecules that encode peptides derived from proteins known to be expressed by a given cell type of interest. In some such embodiments, the nucleic acid molecules in the library comprise, or consist of, nucleic acid molecules that encode peptides known, or predicted, to bind to or be cross-reactive with TCRs or TCR like molecules. In some such embodiments, the nucleic acid molecules in the library comprise, or consist of, nucleic acid molecules that encode peptides that are known to, or predicted to, bind to a defined TCR or TCR like molecule. In some such embodiments, the nucleic acid molecules in the library comprise, or consist of, nucleic acid molecules that encode peptides that are known to, or predicted to, be cross-reactive with a defined TCR or TCR like molecule.

The libraries provided by the present invention can be either “focused” libraries or “random” libraries—depending on their intended use. For example, if the library is to be used to identify epitopes that cross-react with a known T-cell, TCR or TCR-like molecule, a focused library can be generated and used to maximize the chance of finding cross-reactive epitopes. However, in embodiments where there is no prior knowledge of the epitopes that might be identified, a random library (i.e. a library containing randomly generated or randomly selected peptides) may be preferable.

In those embodiments where a “focused” library is to be used, the process of selecting peptides for inclusion in the library will depend on the biological question to be addressed. For example, in one embodiment, where the aim is to identify endogenously presented human epitopes that can bind to or cross react with a known T cell or TCR or TCR-like molecule, one can identify (for example using available sequence databases and sequence analysis tools) all MHC-I ligand sized subsequences present in a mammalian (e.g. human) genome, or in a mammalian (e.g. human) exome, and include as many of those subsequences in the library as possible. In some embodiments, the subset of sequences to include in the library can be further limited by selecting (for example using available sequence analysis tools), either (a) a subset of such sequences predicted to have a given affinity to MHC-I, or (b) a subset of such sequences having similarity to the original target of the T cell, TCR, or TCR-like molecule, or (c) a subset of such sequences known or predicted to be presented on a cell type of interest, and/or by using any other suitable criteria or combination of criteria to select a subset of sequences for inclusion in the library.

In another example, where the aim is to identify viral epitopes that cross react with a known T cell, TCR, or TCR-like molecule, one can identify (for example using available sequence databases and sequence analysis tools) all MHC-I ligand sized subsequences in a viral genome, or in a viral exome, and include as many of them as possible in the library. In some embodiments, the subset of sequences to include in the library can be further limited by selecting (for example using available sequence analysis tools), either (a) a subset of such sequences predicted to have a given affinity to MHC-I, or (b) a subset of such sequences from a particular virus sub-type or strain, or (c) a subset of such sequences from a particular subset of viral proteins.

Similarly, where the aim is to identify epitopes from a certain microbe that cross react with a known T cell, TCR, or TCR-like molecule, one can identify (for example using available sequence databases and sequence analysis tools) all MHC-I ligand sized subsequences in the genome of the microbe, or in the exome of the microbe, and include as many of them as possible in the library. The subset of sequences to include in the library can be further limited by selecting (for example using available sequence analysis tools), either (a) a subset of such sequences predicted to have a given affinity to MHC-I, or (b) a subset of such sequences from a particular microbe sub-type or strain, or (c) a subset of such sequences from a particular subset of proteins expressed by that microbe.

Any suitable constraints can be used to generate peptides for inclusion in the focused libraries of the invention. For example, if there is some prior knowledge of consensus epitopes, or specific amino acid residues that are believed to be important for TCR binding, one can keep those positions constant (i.e. as “anchor” amino acids) and vary all the other positions in the various peptides with 19 different amino acids, or replace each of the other positions with one that has similar chemical features (e.g. in terms of whether they are hydrophobic, hydrophilic, basic, acidic, neutral, etc.), or replace the other positions with one having different chemical features to see if/how that might affect binding.

### Vectors

In some embodiments, the present invention provides vectors comprising the nucleic acid molecules and/or libraries described above and/or elsewhere herein. Any suitable vector can be used, depending on the desired purpose. For example, for cloning and nucleic acid molecule construction purposes, any suitable cloning vector may be used. For expression of ER signal sequence—peptide fusion proteins (peptide fusions) in cells, any suitable expression vector may be used. In some embodiments, the vector is a single-copy competent viral vector. In some embodiments, the vector is a retroviral vector. In some embodiments, the vector is a MSCV retroviral vector.

### Cells

As described above, and elsewhere herein, the present invention provides various methods for screening for and/or identifying T cell epitopes. Such methods involve the use of “engineered target cells.” Engineered target cells are cells that express/display an engineered peptide-MHC (pMHC) complex on their cell surface. The present disclosure describes “PresentER” nucleic acid molecules that, when expressed in cells, result in the generation of engineered peptide-MHC (pMHC) complexes on the cell surface—i.e. producing engineered target cells.

Thus, in some embodiments, the present invention provides a cell comprising a PresentER nucleic acid molecule—as described above and elsewhere herein. Such a cell is an “engineered target cell.” In some embodiments, an engineered target cell may comprise a vector comprising a PresentER nucleic acid molecule. In some embodiments, the present invention provides a population of engineered target cells that comprise a library of PresentER nucleic acid molecules.

In some embodiments, the engineered target cells of the invention are eukaryotic cells. In some embodiments, the engineered target cells of the invention are mammalian cells. In some embodiments, the engineered target cells of the invention are murine cells. In some embodiments, the engineered target cells of the invention are human cells. In some embodiments, the engineered target cells of the invention are human T2 cells. In some embodiments, the engineered target cells of the invention express MHC I. In some embodiments, the engineered target cells of the invention express MHC II. In some embodiments, the engineered target cells of the invention are deficient in one or more components of the cellular antigen presentation machinery. In some embodiments, the engineered target cells of the invention are Tap1-deficient. In some embodiments, the engineered target cells of the invention Tap2-deficient.

The present invention also provides methods for producing “engineered target cells” that that expresses and on their surface an engineered peptide-MHC (pMHC) complex. In some embodiments, such methods comprise culturing a cell comprising a PresentER nucleic acid molecule under conditions that allow for expression of the PresentER nucleic acid molecule. Some such methods also comprise first delivering a PresentER nucleic acid to the cell. Such delivery can be achieved using any suitable method for nucleic acid delivery known in the art, including known transfection methods, viral transduction methods, and the like. Upon expression of the PresentER nucleic acid molecule in the cell, the fusion protein encoded by said nucleic acid molecule is delivered to the endoplasmic reticulum (ER) of the cell. The ER signal sequence portion of the fusion protein will be cleaved from the peptide portion of the fusion protein. The peptide then associates with MHC molecules in the endoplasmic reticulum of the cell forming an engineered peptide-MHC (pMHC) complex. The peptide is not covalently attached to the MHC molecule. The engineered pMHC complex is then be presented/displayed on the surface of the cell. In some embodiments, the cells used to generate the engineered target cells are mammalian cells. In some embodiments, the cells used to generate the engineered target cells are murine cells. In some embodiments, the cells used to generate the engineered target cells are human cells. In some embodiments, the cells used to generate the engineered target cells are human T2 cells. In some embodiments, the cells used to generate the engineered target cells express MHC I. In some embodiments, the cells used to generate the engineered target cells express MHC II. In some embodiments, the cells used to generate the engineered target cells are deficient in one or more components of the cellular antigen presentation machinery. In some embodiments, the cells used to generate the engineered target cells are Tap1-deficient. In some embodiments, the cells used to generate the engineered target cells are Tap2-deficient.

### Kits

In some embodiments, the present invention also provides kits useful in carrying out the various methods described herein. Such kits may comprise any combination of the various different compositions described herein, including nucleic acid molecules, vectors, viruses, peptides, libraries, and cells. Such kits may optionally also comprise instructions for carrying out the methods described herein. For example, in one embodiment the present invention provides a kit for useful in screening for and/or identifying T cell epitopes, the kit comprising a PresentER cloning cassette. In another embodiment, the present invention provides a kit for useful in screening for and/or identifying T cell epitopes, the kit comprising a PresentER nucleic acid molecule. In some embodiments, such kits may comprise one or more oligos or primers useful in construction of PresentER nucleic acid molecules and/or insertion of peptide-encoding sequences into PresentER cloning cassettes, such as one of the specific oligos or primers described herein. In some embodiments, such kits may comprise one or more oligos or primers useful for isolating, amplifying, analyzing, or sequencing peptide-encoding sequences present in a PresentER nucleic acid molecule, such as one of the specific oligos or primers described herein. In some embodiments, such kits may comprise one or more cell types into which PresentER nucleic acid molecules can be delivered to generate engineered target cells. Such cell types may be, for example, mammalian cells (such as murine or human cells). In some embodiments, the cells may be human T2 cells. In some embodiments, the cells may express MHC I. In some embodiments, the cells may express MHC II. In some embodiments, the cells may be deficient in one or more components of the cellular antigen presentation machinery, such as Tap1 and/or Tap2.

### Example

As proof of principle for the invention described herein, experiments were performed to identify cross-reactive epitopes of ESK1⁸ and Pr20⁹, which are TCR mimic (TCRm) antibodies specific to HLA-A*02:01 in complex with a peptide from the WT1 oncogene (WT1 aa126-134 i.e. RMFPNAPYL (SEQ ID NO. 36)—which may be referred to herein using the abbreviation “RMF”) and with a peptide from the tumor associated antigen PRAME PRAME300-309 i.e. ALYVDSLFFL (SEQ ID NO. 37)—which may be referred to herein using the abbreviation “ALY”), respectively. From alanine scans and a crystal structure of ESK1 in complex with its target¹⁰, we knew that several cross-reactive epitopes existed for these antibodies. However, we did not have a cost-effective mechanism to screen the many thousands of other possibly cross-reactive peptides found in the human genome. Therefore, as a proof of the feasibility of this invention, we made a library of 12,500 antigen minigenes, each of which encoded a different 9 or 10 amino acid peptide that is found in the human proteome and, based on our preliminary data, might be cross-reactive with either ESK1 or Pr20. The library was designed based on known sequences that bound to theses TCRms. The human exome was searched for similar sequences to these known sequences. For instance, we knew that the “R” and “P” in the 1^{st} and 4^{th} position of RMFPNAPYL (SEQ ID NO: 36) were important for binding to ESK1 (10). Therefore, we searched the exome for peptides with an “R” in position 1 and a “P” in position 4 and included those sequences in our library. It should be noted that while in this Example a “focused” library was made based on known sequences, non-focused random libraries could also be generated and used—for example encompassing any sequence in the exome, or encompassing any sequence in any genetic code from any organism, or simply any random sequence. Minigenes encoding these antigens were generated and cloned into the PresentER framework. In a pooled screen of ESK1 cross-reactive targets we identified several known ESK1 binders as well as over 200 cross-reactive epitopes. Such cross-reactive epitopes could be used to define the specificity of TCRs or TCRms, for example in order to predict possible toxicities of therapeutic agents or to facilitate the design of improved therapeutic agents.

Furthermore, as demonstrated further below, various different peptides identified using the compositions and methods of the present invention could be recognized by fluorescently labeled TCRs, and could potently stimulate T cells in vitro and mediate cytotoxicity in vivo.

Cloning of the MMTV1 Antigen Minigenes

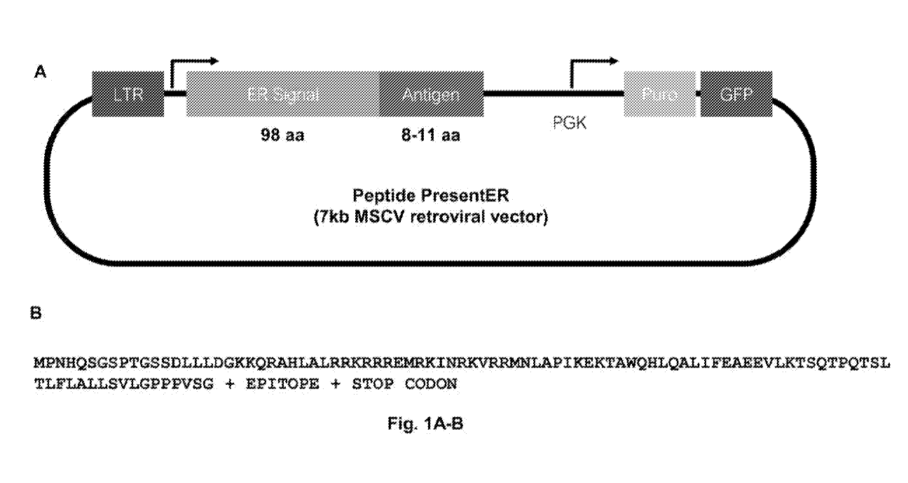

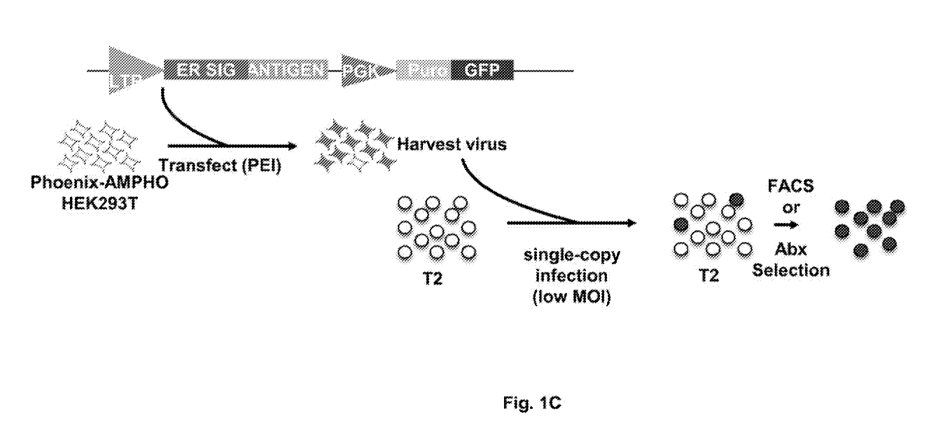

All cloning was performed according to standard procedures. The MLP vector is the “MSCV-LTRmiR30-PIG” vector described in Dickins 2005 Nature Genetics. A related MSCV vector known as “PIG”, which could be used in place of the MLP vector, is commercially available from Addgene (Addgene plasmid no. 18751; www.addgene.org/18751/). We constructed the first version of the antigen minigene using gBlocks (GBLOCKS) gene fragments (double-stranded DNA, sequence-verified genomic blocks) from Integrated DNA Technologies (IDT; www.idtdna.com) containing Xhol and EcoRI restriction sites surrounding the MMTV gp70 ER targeting sequence (ENV MMTVC obtained from the internet signal peptide database found at http://signalpeptide.de/; henceforth known as MMTV1) upstream of either the sequence encoding RMFPNAPYL (SEQ ID NO. 36) or ALYVDSLFFL (SEQ ID NO. 37) followed by a stop codon (FIG. 1A-B). MLP was digested with Xhol and EcoRI for 1-4h at 37° C., treated with Calf Intestinal Phosphatase for 30m and then purified on an agarose gel. The gBlocks containing the MMTV ER targeting sequencing and antigen were amplified with the following oligonucleotides: F: 5′ AATTCACTGACTGACTGACTGAACA 3′ (SEQ ID NO. 38) R: 5′ GTGATTCGGTCAGTTGTTGTACG 3′ (SEQ ID NO. 39). Amplicons were PCR purified, digested with Xhol/EcoRT and then PCR purified again. Insert and vector were ligated with T4 ligase overnight at 16° C. and transformed into NEB Stable cells. Single bacterial colonies were selected and miniprepped.

Expression and Purification of Retrovirus

All retroviral generation was performed according to standard protocols. HEK293T amphoteric cells were seeded onto 10 cm or 15 cm plates and grown until 70% confluence. Cells were transfected with 45 μg Polyethylenimine (PEI) (stock: 1 μg/μ1) and 15 μg of plasmid DNA (10 cm plates) or 25 μg plasmid DNA and 750 PEI (15 cm plates). Viral supernatant was harvested every 12h until 72h post-transfection. Supernatant was kept at 4° C. at all times. After the final harvest, viral supernatant was spun down at 500×g for 10m to remove any cells and the supernatant was pooled. Viral supernatant was either used immediately or concentrated with Clontech's RetroX concentrator, flash frozen and stored at −80° C. (FIG. 1C).

Spinoculation

T2 cells (ATCC CRL1992) were obtained from ATCC. T2 cells are human lymphocyte cells that do not express HLA DR and are Class II major histocompatibility (MHC) antigen negative and TAP deficient. Cultures of T2 cells were maintained in 10% FBS/RPMI and split 1:5 every 3-4 days. Cells were tested weekly or monthly for mycoplasma contamination. Healthy, growing T2s were spinoculated at 2,000×g for 2h at 25° C. in 6-well format in a bucket centrifuge with 4 μg/ml polybrene and variable amount of virus (depending on titer). T2s were allowed to recover for several hours at 37° C. and then fresh media was added.

ESK1 and Pr20 Labeling and Staining

ESK1 and Pr20 monoclonal antibodies were fluorescently labeled with the Innova Biosciences Lightning Link (LIGHTNING LINK) kit according to the manufacturer's instructions. After labeling, antibodies were tittered on T2 cell pulsed with cognate peptide (RMFPNAPYL (SEQ ID NO. 36) or ALYVDSLFFL (SEQ ID NO. 37)). Soluble peptides were pulsed onto T2 cells in culture at 20 μg/ml overnight. Antibody staining was performed according to standard protocols. Briefly, the staining protocol is (1) harvest cells, (2) wash 2× with ice cold PBS, (3) block for 10 minutes at room temperature with 10% Fc Block, (4) Add antibody at appropriate concentration to cells, (5) Wash 2× with ice cold FACS buffer (0.01 NaN₃, 5% FBS, PBS), (6) resuspend in FACS buffer+DAPI.

pMHC are Specifically Encoded by the PresentER Minigene

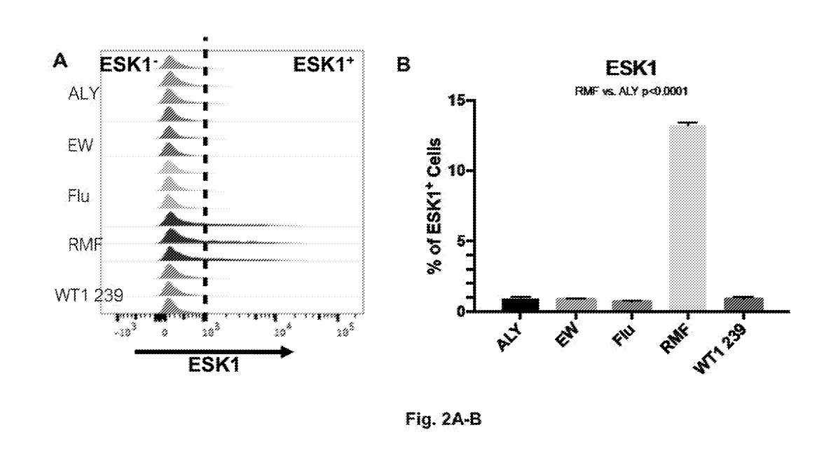

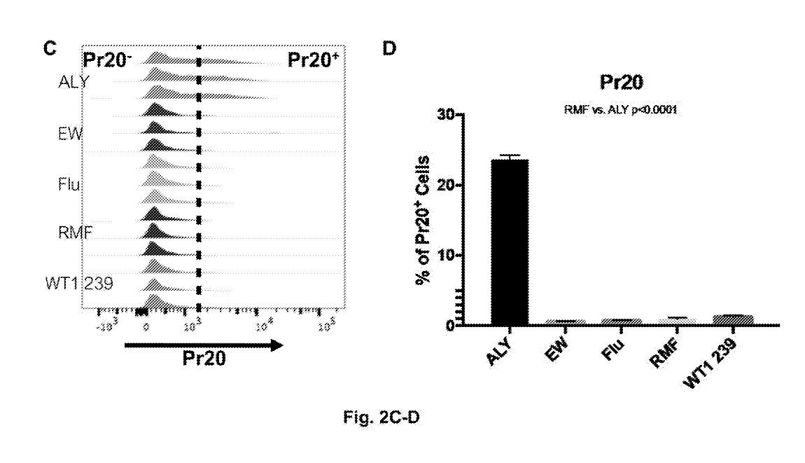

Minigene expressing T2 cells stained with fluorescently labeled ESK1 and Pr20 and analyzed by flow cytometry showed that ESK1 bound only to cells expressing the RMF minigene whereas Pr20 preferentially bound to cells expressing the ALY minigene (FIG. 2). Neither Pr20 nor ESK1 bound to PresentER minigenes expressing three other irrelevant antigens. These results demonstrate that the Peptide PresentER system generated specific pMHC at levels sufficient to detect using fluorescently labeled TCRm antibodies by flow cytometry.

In order to test whether the endoplasmic reticulum targeting sequence was necessary for generating pMHC, we cloned two scrambled versions of the ER signal sequence and tested whether peptide antigens downstream of these scrambled sequenced could present pMHC.

Cloning a Scrambled WTV2 Targeting Sequencing

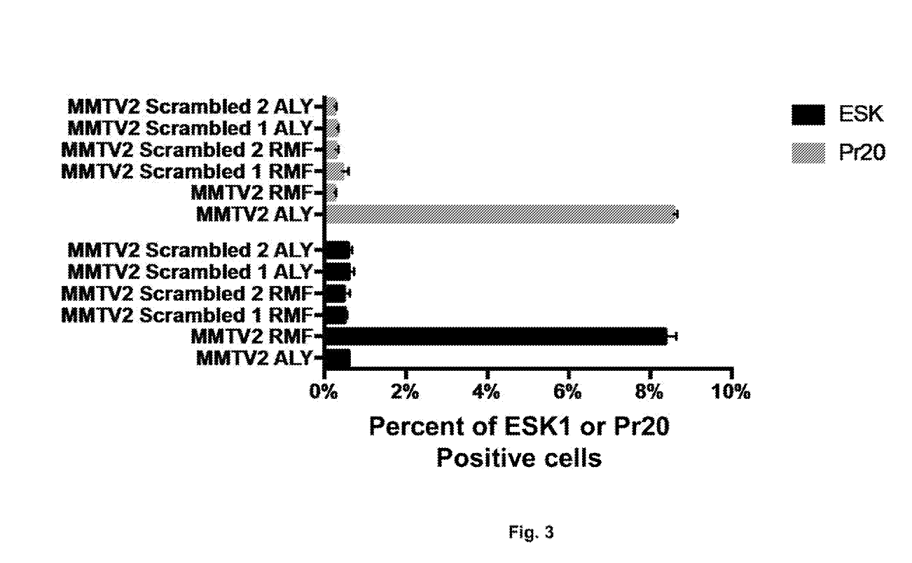

IDT GBLOCKS containing scrambled ER signal sequences were synthesized, digested and ligated into MLP as before. Scrambled ER targeting sequences are included below. Vectors containing these minigenes were used to generate retrovirus and transduce T2 cells. Only T2 cells transduced with minigenes utilizing a non-scrambled ER signal sequence generated pMHC that could be detected with ESK1 or Pr20 (FIG. 3).

T Cell Receptors Bind to Cells Transduced with PresentER Minigenes

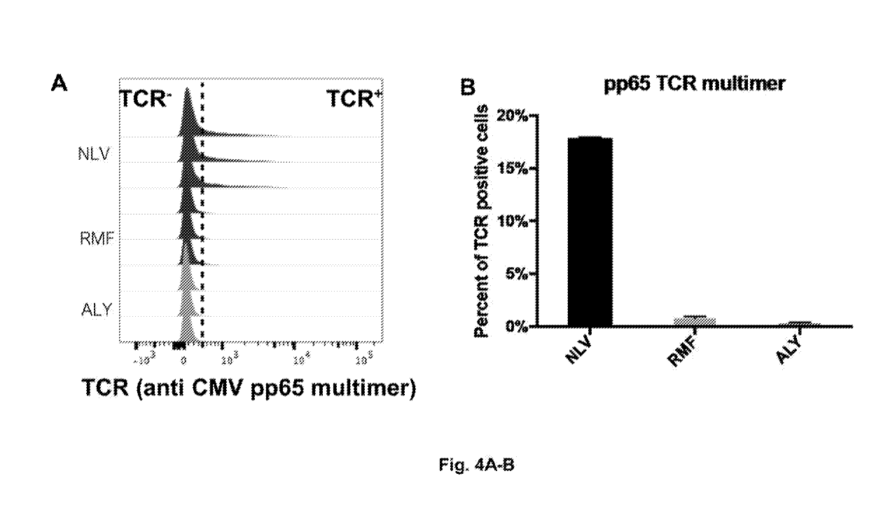

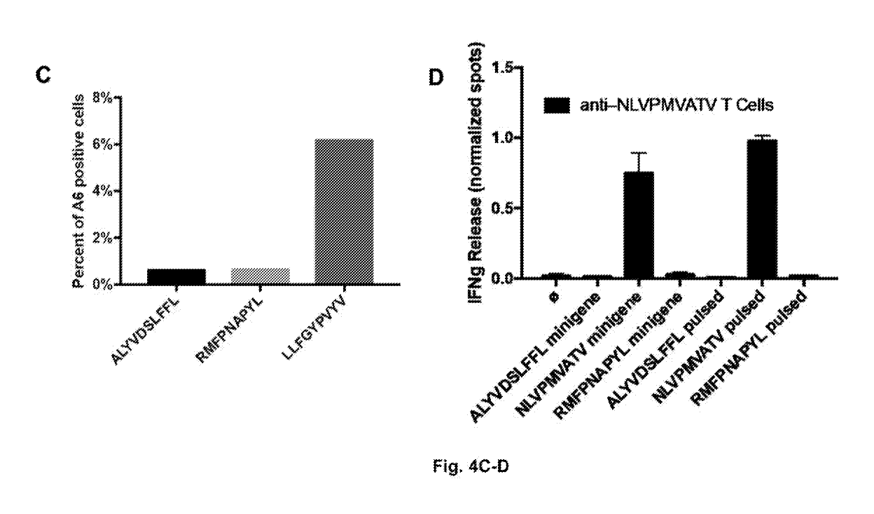

With strong evidence that TCRm antibodies could bind to peptides expressed using PresentER, we turned to a soluble T cell receptor. We obtained a fluorescently labeled TCR multimer from Altor Biosciences specific for cytomegalovirus pp65 aa495-503 (NLVPMVATV SEQ ID NO. 47). We stained T2 cells expressing “RMF”, “CMV” (see below for details) or “ALY” peptides and noted that only NLV expressing cells were bound by the soluble TCR (FIG. 4A-B).

In preparation for performing a library screen of a human TCR, we obtained plasmids encoding the A6 TCR (17) alpha and beta chains. This TCR is very well studied with many known binding and non-binding peptides. We modified the DNA sequence encoding the beta chain to include a tag—AviTag (AVITAG) (GLNDIFEAQKIEWHE SEQ ID NO. 45 and ggcctgaacgatatttttgaagcgcagaaaattgaatggcatgaa SEQ ID NO. 46)—at the C terminus using the Q5 mutagenesis kit from New England BioLabs (NEB) and two oligos (A6b_AviTag_F gcagaaaattgaatggcatgaaTAAGCTTGAATTCCGATCCGG (SEQ ID NO. 48) and A6b_AviTag_R gcttcaaaaatatcgttcaggccGTCTGCTCTACCCCAGGC SEQ ID NO. 49). The addition of the AviTag allows the beta chain to be biotinylated in vivo when bacteria are co-transformed with a plasmid encoding the BirA enzyme (Addgene plasmid no. 26624; https://www.addgene.org/26624/). Vectors encoding the alpha and beta chains were separately transformed into BL21(DE3) competent cells (NEB product #c2527) and grown under standard bacterial growth conditions. The beta chain vector was co-transfected with the vector encoding BirA. When bacterial density reached an OD of >0.7, Isopropyl β-D-1-thiogalactopyranoside (IPTG) was added to 1 mM to induce expression of the alpha and beta chains. The growth media for cells expressing the AviTagged beta chain was additionally supplemented with 0.5 mM D-biotin. Bacteria were grown for 30 hours and inclusion body purification was performed using standard protocols. The two denatured chains were mixed together in 1 liter of refolding buffer (50 mM Tris-HCl, 2.5M Urea, 2 mM NaEDTA, 0.74 g/L cysteamine, 0.83 g/L cystamine, 0.2 mM PMSF, pH of 8.15) and incubated overnight at 4° C. The refolding buffer was then dialyzed against 10 mM Tris for >30 h in 7 kd cut-off snakeskin dialysis tubing. Refolded protein was concentrated on a DEAE anion exchange column and size-selected by FPLC. Finally, the refolded/biotinylated A6 TCR was conjugated to a Streptavidin R-Phycoerythrin Conjugate (Life Technologies (100187-WEB)) to generate tetramers and used to stain T2 cells encoding PresentER “ALY,” “RMF,” or “LLF” (FIG. 4C). The A6 TCR specifically bound to T2 cells encoding “LLF” which is its target antigen.

Generation of Antigen-Specific T Cells and IFN-Gamma Release Assay

Just because a soluble TCR could bind to NLV expressing cells does not mean that the PresentER system could activate an actual T cell. Therefore, experiments were performed to determine if the PresentER system could activate an actual T cell. Peripheral blood mononuclear cells (PBMCs) were obtained from a donor and expanded according to standard protocols (12). T cells were repeatedly stimulated with HLA-A*02/NLVPMVATV (NLVPMVATV—SEQ ID NO. 47—is an HLA-A*02 epitope from CMVpp65) to select a polyclonal, antigen-specific population. These CMV peptides were used for these “proof of concept” experiments because T cells against CMV can readily be generated from normal healthy donors. Therefore, we could rapidly and consistently generate T cells that react to cells presenting these epitopes. Moreover, since CMV is a commonly used epitope—many molecules have been developed that bind to this pMHC, such as the Altor Biosciences CMV multimer described above. An IFN-gamma release assay was performed by incubating T cells with target cells overnight in a 96-well filtration plate and performing an ELISPOT for IFN-gamma. Target cells were either pulsed with 20 μg/ml of soluble peptide or had previously been transduced with PresentER minigenes.

Anti-NLV T cells released IFNg only when challenged with T2s that had been pulsed with NLV or transduced with a PresentER minigene encoding NLV (FIG. 4D).

These results demonstrate: (1) that PresentER can be bound by soluble, multimerized TCRs, and (2) that PresentER can stimulate T cells to release IFN gamma.

The PresentER System is Single-Copy Competent

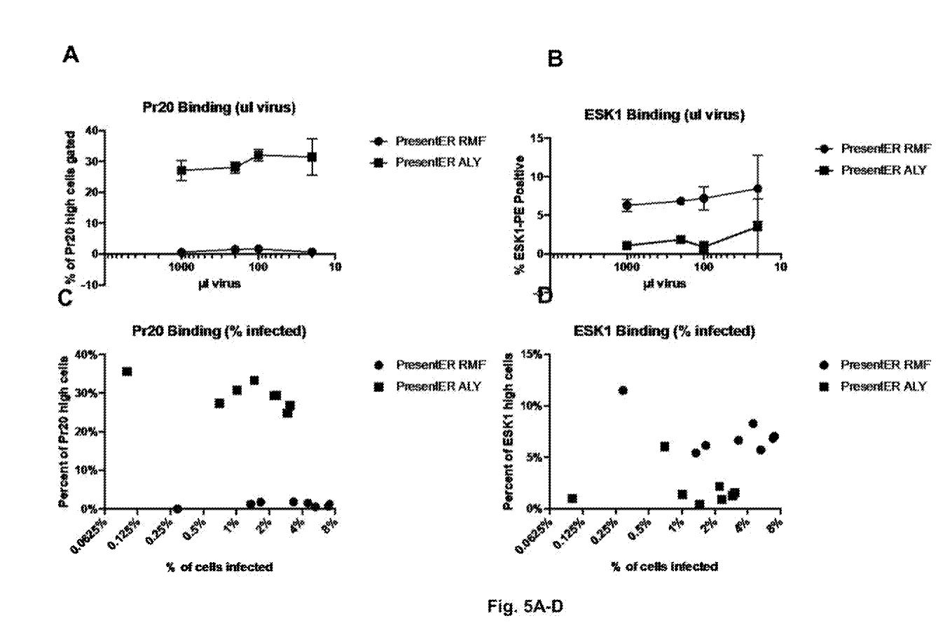

In order to perform pooled library screening, sufficient peptide must be produced from a single minigene copy per cell¹³. If multiple copies of a minigene are necessary for a phenotype to be detectable, then pooled library screening is not possible. In order to confirm that the PresentER system is single-copy competent, we started with a low multiplicity of infection (MOI) viral supernatant and spinoculated T2 cells with serial dilutions of the viral supernatant. As the concentration of viral particles is decreased by 10-fold, the number of cells with multiple minigene copies will decrease by 100-fold. ESK1 and Pr20 binding were assessed for each of the spinoculated cell cultures. Across ˜100-fold dilution of viral supernatant, the level of ESK1 and Pr20 binding remained very similar (FIG. 5A-B). Similarly, the fraction of GFP positive cells that bound to Pr20 and ESK1 remained the same across cultures with an order of magnitude fewer GFP positive cells (i.e. 10× lower MOI corresponds to 100× fewer cells with double integrands) (FIG. 5C-D). Therefore, we conclude that a single copy of the PresentER minigene generates numerically sufficient pMHC molecules to be detected by fluorescently labeled TCRm antibodies.

The PresentER System is Designed for Cost-Effective Library Cloning and High Throughput Sequencing (HTS)

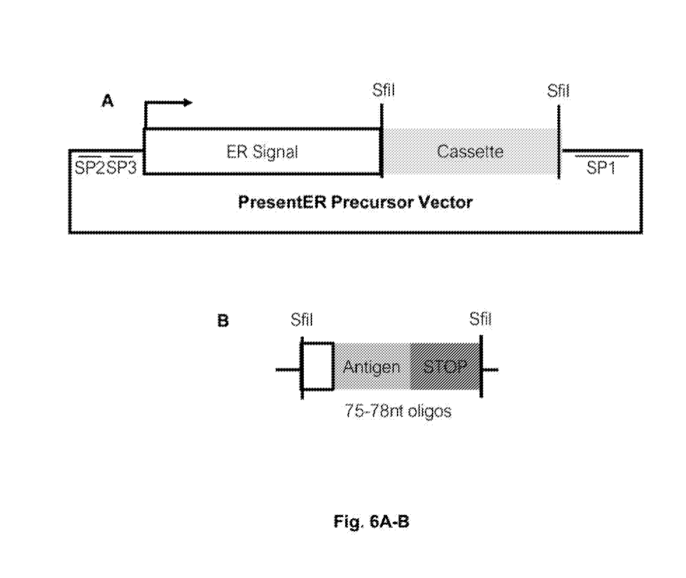

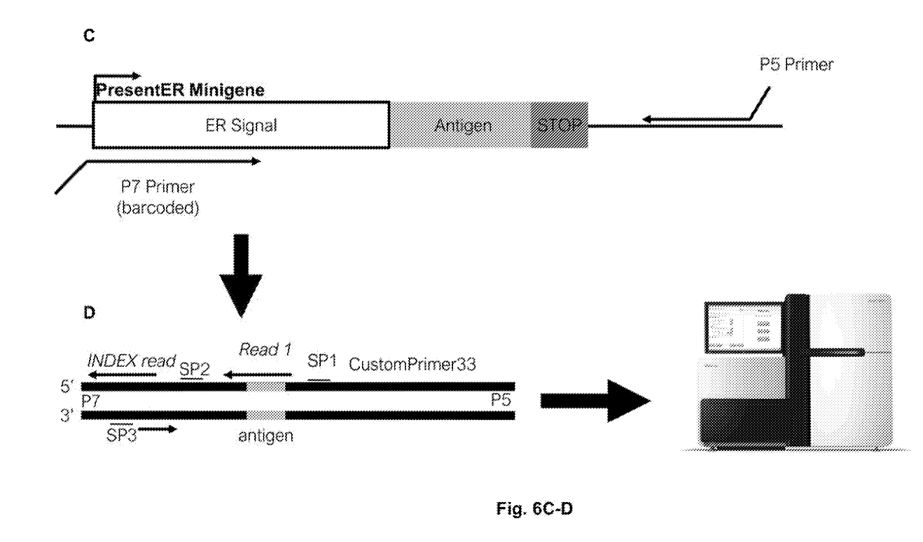

We re-architected the MSCV vector to enable cost-effective cloning and high throughput sequencing. The MMTV gp70 ER signal sequence was modified to include a C-terminal SfiI restriction digest site and a downstream removable cassette with another SfiI restriction digest site (FIG. 6A). The modified ER signal sequence did not impact pMHC presentation.

Cloning of a Modified MMTV2 Cassette into MLP

A GBLOCK containing a modified ER targeting sequencing followed by a 200 nt cassette was amplified, digested and ligated into MLP (FIG. 6A). The ER targeting sequence was modified to include a SfiI restriction site at the C-terminus. Furthermore, the vector was modified to include Illumina signal sequences: P5 and P7 hybridization sites along with SP1, SP2 and SP3 primer binding sites. The final amino acids of the gp70 targeting sequence were modified as follows: L T L F L A L L S>A V L G>A P P P V S G (SEQ ID NO: 50). (i.e. L T L F L A L L S V L G P P P V S G (SEQ ID NO. 51) was change to L T L F L A L L A V L A P P P V S G (SEQ ID NO. 52). The modified targeting sequencing (SEQ ID NO. 52) is known as MMTV2. The amino acid changes were made to introduce SfiI cloning sites. The cloned cassette was digested with SfiI, treated with CIP (Calf Intestinal Phosphatase) and gel purified according to standard molecular cloning protocols. Cloning of a 24-33 nt peptide antigen (8-11 amino acid) into the vector backbone is accomplished by synthesizing a short oligonucleotide (72-81 nt) with SfiI digestion sites, the final amino acids of the ER signal sequence and the antigen followed by a stop codon (FIG. 6B). The cloned PresentER minigene is comprised of the ER signal sequence, followed immediately by the antigen and terminated with a stop codon (FIG. 6C). Finally, amplification of the minigene with barcoded primers using the plasmid or genomic DNA as template yields an Illumina-sequencing compatible amplicon (FIG. 6D).

Cloning WTV2 Antigens into the MLP/MMTV2 Cassette

Oligonucleotides for several peptides were ordered from IDT with the following format:

GGCCGTATTGGCCCCGCCACCTGTGAGCGGG (SEQ ID NO. 40)+ANTIGEN+TAAGGCCAAACAGGCC (SEQ ID NO. 41—includes TAA stop codon—but other stop codons could also be used).

Oligos corresponding to the following peptides were cloned: RMFPNAPYL (SEQ ID NO. 36—“RMF”), ALYVDSLFFL (SEQ ID NO. 37—“ALY”), NLVPMVATV (SEQ ID NO. 47—“NLV”), (QLQNPSYDK SEQ ID NO. 42—“EW”), (GILGFVFTL SEQ ID NO. 43—“Flu”), and (NQMNLGATL SEQ ID NO. 44—“WT1” 239). Oligos were PCR amplified with T7 SfiI and T3 SfiI, digested with SfiI, PCR purified, ligated into the PresentER plasmid and NEB Stable cells were transformed with the ligand products.

Synthesizing a Library of Possible Peptide Targets of Pr20 and ESK1

In the course of developing Pr20 and ESK1, we validated the antibody specificity by individually testing peptides that might be cross-reactive with the two epitopes. For ESK1, an arginine in the 1^{st} position and a proline in the 4^{th} position was strongly preferred. For Pr20, the specificities were much less clear. We searched the human proteome for peptides that looked similar to the target epitopes of ESK1 and Pr20 and were predicted to bind to HLA-A*02:01. In this example of a PresentER library, we first downloaded all of the proteins found in the human genome from UniProt. Then we found every 9-10 amino acid long subsequence and calculated its predicted affinity to HLA-A*02:01 using NetMHCPan (http://www.cbs.dtu.dk/services/NetMHCpan/). Finally, we selected those peptides that fit the motifs presented below in Table 2. Additionally, we included all single amino acid changes to RMF and ALY. In another embodiment, libraries of random peptides can be used. In another embodiment, libraries of viral or bacterial peptides can be used. We discovered 1,200 RMF-like epitopes and 24,500 possible ALY-like epitopes.

TABLE 2

| | 1 | 2 | 3 | 4 | 5 | 6 | 7 | 8 | 9 | 10 |
|---|---|---|---|---|---|---|---|---|---|---|
| ESK1 Target | R | M | F | P | N | A | P | Y | L | |
| (SEQ ID NO: 36) | | | | | | | | | | |
| ESKI Library | R | * | * | P | * | * | * | * | * | |
| Pr20 Target | A | L | Y | V | D | S | L | F | F | L |
| (SEQ ID NO: 37) | | | | | | | | | | |
| Pr20 Library | * | * | * | [D, E] | [K, H, | [K, H, | [K, H , | F, W, Y, | F, W, Y, | L, V, I |
| | | | | | R] | R] | R] | V, L, I | V, L, I | |

Table 2 shows the amino acid residues allowed in each position (positions/columns 1-10) for human proteome peptides included in the library. Permitted residues are shown without parentheses/brackets. Non-permitted residues are shown in square parentheses/brackets. Asterisks (*) denote positions where any residue is allowed.

Design of a PresentER Library for ESK1 and Pr20 Targets

All single amino acid changes to ESK1 and Pr20 were included in the library, along with known binders and non-binders to ESK1 and Pr20. A consensus sequence was generated for ESK1 and Pr20 based on pre-existing ESK1 and Pr20 binding assay data (Table 2). Peptides found in the human proteome that matched the consensus were considered for inclusion in the library. A final library containing all of the ESK1 RMF-like peptides and a randomly-selected subset of the ALY-like peptides was created. We ordered a pool of 12,500 oligonucleotides from CustomArray, corresponding to all the RMF-like peptides we found in the human genome and about half of the ALY-like epitopes along with positive and negative control minigenes and all single amino acid mismatches of RMF and ALY (Table 3).

TABLE 3

| Category | # Constructs (%) |
|---|---|
| Positive and Negative Controls | 13 (0.1%) |
| ESK1 amino acid scan (all 1 AA mismatch) | 180 (1.45%) |
| Pr20 amino acid scan (all 1 AA mismatch) | 190 (1.5%) |
| ESK1 genomic off-targets | 1,157 (9.3%) |
| Pr20 genomic off-targets | 10,893 of 24,500 (87.6%) |
| Total | 12,433 |

Table 3 shows the number of peptides (constructs) matching each of five categories (Positive/Negative controls, ESK1 amino acid scans, Pr20 amino acid scans, ESK1 genomic off-targets, and Pr20 genomic off-targets).

Cloning the First PresentER Library

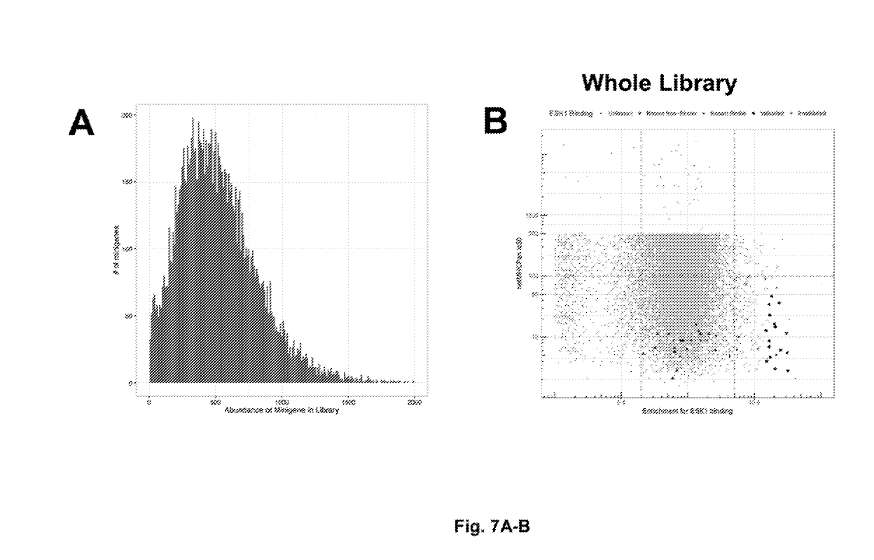

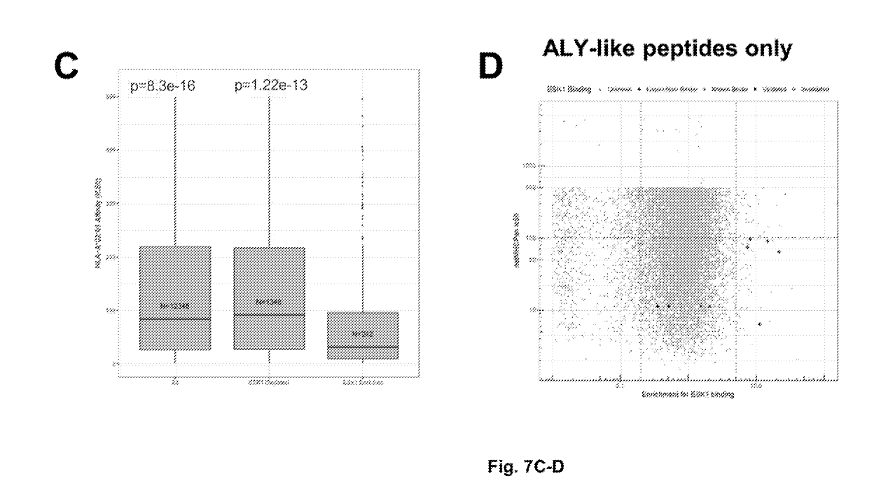

Cloning of the PresentER library was performed according to standard library cloning methods. A brief description of the cloning is as follows. A soluble oligonucleotide pool was ordered from CustomArray with 12,472 individual oligonucleotides. The pool was aliquoted and then diluted to 5 ng/μl. Twelve identical PCR reactions were performed to amplify the pool with the T7_SfiI and T3_SfiI primers. Amplification was visualized on a gel. Amplicons were pooled and PCR purified with Qiagen's MinElute (MINELUTE) kit. Purified amplicons were digested in triplicate with SfiI until the digestion product could be visualized on an analytic gel—and then pooled and again purified using the MinElute kit. Digested amplicons were ligated overnight at 16° C. into the pre-digested PresentER backbone in 6 separate ligations with high concentration T4 ligase (2×10⁶ units/ml), 300 ng backbone and 20 ng of insert per reaction. Two insert-negative ligations were included in order to calculate the nonspecific ligation rate. The 6 ligations containing insert were pooled together, as were the 2 ligations without insert. A test transformation of 1 μl of ligated insert vs. no-insert control was performed with NEB Stable cells and 1:10, 1:100 and 1:1000 dilutions were plated. Several colonies were visualized on the insert positive plates but no colonies were visualized on the insert negative plate. 15 colonies were picked, miniprepped and sequenced to check if ligation worked. Ligation products were split into 6 tubes and DNA was precipitated and concentrated using phenol/NaOAc/EtOH precipitation and resuspended in 20 μl EB. DH5-alpha electrocompetent cells were electroporated with 20 of ligation product and recovered in 1 ml of SOC for 1 h. 100 of transformed insert positive and negative cells were serially diluted out to 1:1×10⁶ and plated on 10 cm ampicillin plates. The remainder of the bacteria was plated on 4×15 cm ampicillin plates. After overnight growth, the number of colonies was calculated at 46×10⁶, which is >1,000× average minigene representation. Plates were scraped into 300 ml of TB+ampicillin and shaken for 3.5h at 37° C. and then maxiprepped to yield 1.1 mg of DNA. The library was amplified from plasmid DNA using the P5 and P7 Index #1 primers (below) and submitted for diagnostic sequencing on the HiSeq (FIG. 7A).

Library Screen, Genomic DNA Purification, Amplification and Illumina Sequencing

Retrovirus containing the PresentER minigene library was produced by transfection of HEK293T phoenix amphoteric cells and viral supernatant was tittered on T2 cells. Two hundred and thirty million T2 cells were spinoculated with the PresentER library at an MOI of less than 1 (˜13% infected). Cells were expanded for two days and then GFP positive cells were sorted by Flow activated cell sorting (FACS). After sorting, cells were cultured in 2× penicillin/streptomycin media overnight. The number of live, infected cells was maintained at >12.5×10⁶ at all times in order to maintain an average of >1000× representation of each minigene. After several days of growth, the T2 cells were viably frozen in several aliquots that could be used for repeated experiments. Before performing a library screen, cells were thawed and cultured for several days before being split into two batches and each batch split into a further 2 replicates (4 samples total). Two of the replicates were washed and frozen and represent the “background/unsorted” library. The other two replicates were stained with DAPI and either of the two TCRm: ESK1 or Pr20. The replicates were sorted by FACS based on the signal of DAPI, GFP and the TCRm. Gates for TCRm “high” and “low” samples were selected by comparing the relative TCRm staining levels of T2s spinoculated with single PresentER minigenes (RMF, ALY and NLV). This sorting protocol yields four samples: (a) TCRm High #1, (b) TCRm High #2, (c) TCRm Low #1, (d) TCRm Low #2. After sorting, cells were washed and frozen. DNA was purified from sorted cells with the Qiagen Gentra Puregene Cell Kit.

PresentER minigenes were PCR amplified from genomic DNA using the NEB Phusion High-Fidelity polymerase with barcoded P7 and P5 primers (see DNA Sequences). All PCR reactions were prepared in a DNA and DNAse free PCR hood that was regularly cleaned and maintained contamination free. DNA amplicons of ˜436 nt in length were gel purified and submitted for sequencing on an Illumina HiSeq using Illumina SP1 or a custom sequencing primer that begins sequencing after the amplicon's constant region (MMTVCustomPrimer33).

Enrichment for the ESK1 or Pr20 TCRm was calculated for each minigene as the ratio of its abundance in the TCRm binding sorted samples versus the TCRm non-binding samples, normalized by the abundance in the unsorted library. Furthermore, for each peptide encoded by the minigene, we calculated the expected affinity to HLA-A*02:01 with NetMHCPan¹⁴. The affinity of each peptide to HLA is reported as the half-maximal inhibitory concentration (IC₅₀), therefore smaller numbers signify higher affinity.

We observed ˜220 minigenes (<2%) with greater than 5-fold enrichment for ESK1 binding (FIG. 7B). The peptides encoded by these minigenes were of higher affinity to HLA-A*02:01 than the peptides across the whole library or in the 5-fold depleted peptides (FIG. 7C). Moreover, although about 10% of peptides were selected for the library because they were possible genomic off-targets of ESK1 or 1 amino acid mismatched to RMFPNAPYL (SEQ ID NO 36) (Table 3), almost 60% of the enriched peptides fit these criteria. Finally, we included several positive and negative control peptides in the library: peptides where we had experimental evidence for their binding to ESK1. None of the negative control peptides (those where we had experimental data showing no binding to ESK1) were enriched for ESK1 binding, whereas several of the known positive controls were enriched for ESK1 binding. These data strongly suggest that our screen identified true ESK1 off targets.

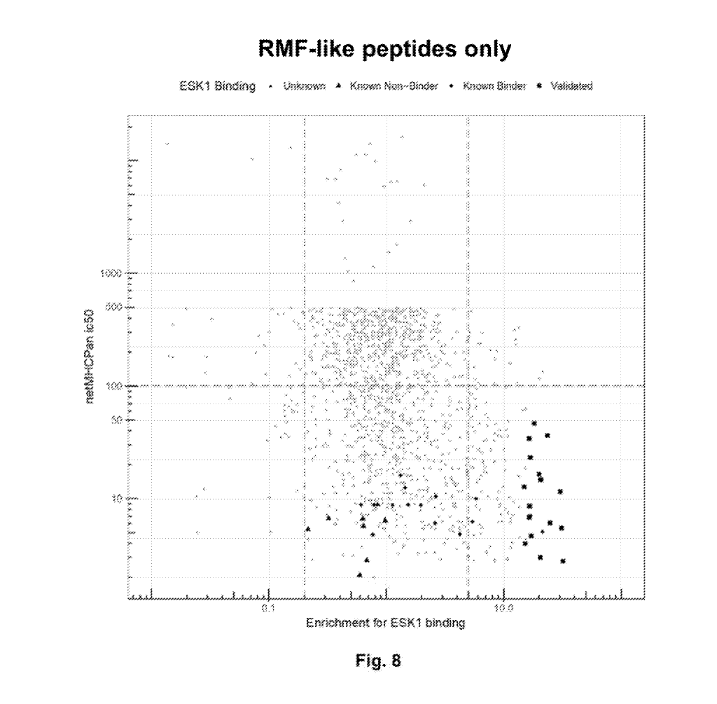

In examining the subset of peptides selected for their similarity to RMFPNAPYL (SEQ ID NO. 36) we noticed that there were approximately equal numbers of peptides enriched and depleted for ESK1 binding when HLA-A*02:01 IC₅₀ was greater than 100 nM. Below 100 nM, there was a striking enrichment for ESK1 binding (FIG. 8). In other words: peptides that bind to ESK1 are much more likely to be found with IC₅₀'s less than 100 nM. As a result, we restricted our analysis of enriched peptides to only those below 100 nM. In total, we identified 222 peptides that are cross-reactive with ESK1.

In order to validate that the peptides discovered in the screen are in fact ESK1 binders, we synthesized 27 of the top hits in addition to 3 positive and 2 negative ESK1 binding control peptides. Peptides were synthesized at microgram scale by JPT Peptide Technologies and resuspended in DMSO followed by dilution in water. Soluble peptide was added to T2 cells in 96-well plate format and incubated overnight. The cells were washed and stained with fluorescently labeled ESK1 as described above. More than 80% of the peptides were found to be ligands of ESK1, thus validating our ability to use the PresentER system to discover novel ligands of TCR mimic antibodies. The validated peptides are marked by squares in FIGS. 7B and 8.

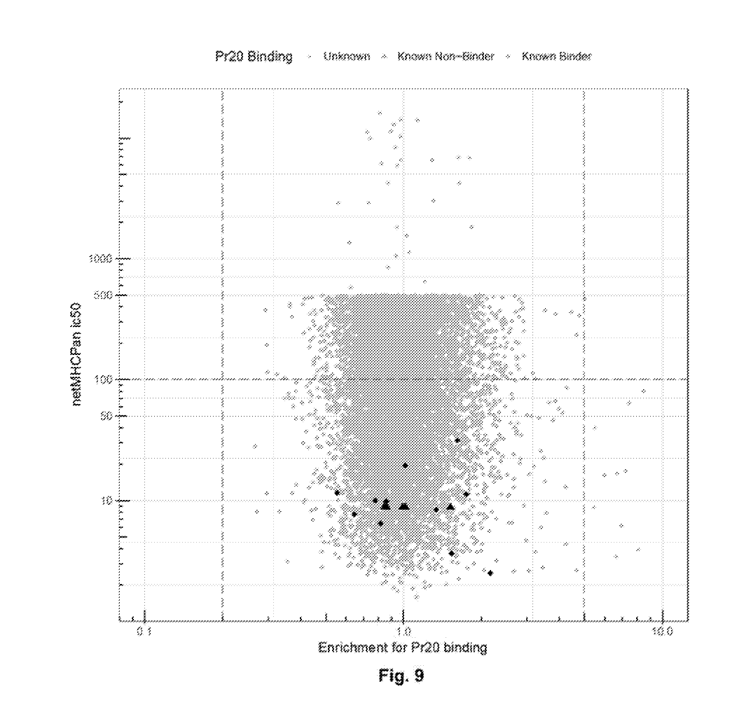

The library screen was repeated with the Pr20 library using the same procedure and conditions that were employed for the ESK1 library screen. We observed 10 minigenes with greater than 5-fold enrichment for Pr20 binding and an ic50<100 nM (FIG. 9).

Bioinformatic Analysis

FASTQ files for each sample were aligned to the DNA sequences of the library with Bowtie2. The number of reads corresponding to each minigene was tabulated using custom R scripts. The relative of abundance of each minigene in each sample was calculated as: (# reads mapping to minigene A)/(# reads mapping to all minigenes). The mean relative abundance was calculated for each pair of replicates and divided by the mean relative abundance in the unsorted samples. ESK1 enrichment was calculated for each minigene as (“ESK1 high” mean relative abundance)/(“ESK1 low” mean relative abundance). Binding affinity to HLA-A*02:01 was calculated using NetMHCPan.

### REFERENCE LIST

1. Chang, A. Y. et al. Opportunities and challenges for TCR mimic antibodies in cancer therapy. 16, 979-987 (2016).
2. Linette, G. P. et al. Cardiovascular toxicity and titin cross-reactivity of affinity-enhanced T cells in myeloma and melanoma. Blood 122, 863-871 (2013).
3. Cameron, B. J. et al. Identification of a Titin-derived HLA-A1-presented peptide as a cross-reactive target for engineered MAGE A3-directed T cells. Sci Transl Med 5, 197ra103-197ra103 (2013).
4. Crawford, F. et al. Use of baculovirus MHC/peptide display libraries to characterize T-cell receptor ligands. Immunol. Rev. 210, 156-170 (2006).
5. Birnbaum, M. E. et al. Deconstructing the peptide-MHC specificity of T cell recognition. Cell 157, 1073-1087 (2014).
6. Bacik, I., Cox, J. H., Anderson, R. & Yewdell, J. W. TAP (transporter associated with antigen processing)-independent presentation of endogenously synthesized peptides is enhanced by endoplasmic reticulum. J. Immunol. 152, 381-387 (1994).
7. Lazoura, E. et al. Non-canonical anchor motif peptides bound to MHC class I induce cellular responses. Mol. Immunol. 46, 1171-1178 (2009).
8. Dao, T. et al. Targeting the intracellular WT1 oncogene product with a therapeutic human antibody. Sci Transl Med 5, 176ra33 (2013).
9. Chang, A. et al. A Therapeutic TCR Mimic Monoclonal Antibody for Intracellular PRAME Protein in Leukemias. Blood 126, 2527-2527 (2015).
10. Ataie, N. et al. Structure of a TCR-Mimic Antibody with Target Predicts Pharmacogenetics. J. Mol. Biol. 428, 194-205 (2016).
11. Van Kaer, L., Ashton-Rickardt, P. G., Ploegh, H. L. & Tonegawa, S. TAP1 mutant mice are deficient in antigen presentation, surface class I molecules, and CD4-8+ T cells. Cell 71, 1205-1214 (1992).
12. Doubrovina, E. S. et al. In vitro stimulation with WT1 peptide-loaded Epstein-Barr virus-positive B cells elicits high frequencies of WT1 peptide-specific T cells with in vitro and in vivo tumoricidal activity. Clin. Cancer Res. 10, 7207-7219 (2004).
13. Fellmann, C. et al. An optimized microRNA backbone for effective single-copy RNAi. Cell Rep 5, 1704-1713 (2013).
14. Hoof, I. et al. NetMHCpan, a method for MHC class I binding prediction beyond humans. Immunogenetics 61, 1-13 (2009).
15. Zhou et al. “TAP2-defective RMA-S cells present Sendai virus antigen to cytotoxic T lymphocytes.” Eur. J. Immunol. 23, 1796-1801 (1993).
16. Townsend et al. “Association of class I major histocompatibility heavy and light chains induced by viral peptides.” Nature 340, 443-448 (1989).
17. Utz, U. et al. Analysis of the T-cell receptor repertoire of human T-cell leukemia virus type 1 (HTLV-1) Tax-specific CD8+ cytotoxic T lymphocytes from patients with HTLV-1-associated disease: evidence for oligoclonal expansion. J Virol 70, 843-851 (1996).
18. Dickins, R. A. et al. Probing tumor phenotypes using stable and regulated synthetic microRNA precursors. Nat Genet 36, 456-1295 (2005).

We claim:

1. A T cell epitope screening method, comprising:
(a) contacting an engineered target cell, or a population of engineered target cells, with a T cell, a TCR, or a TCR-like molecule, and
(b) performing an assay to determine whether the T cell, TCR, or a TCR-like molecule binds to the engineered target cell, or population of engineered target cells, and/or to measure the strength of any such binding,
wherein the engineered target cell(s) comprises a recombinant nucleic acid molecule, or a library of recombinant nucleic acid molecules, wherein the recombinant nucleic acid molecule(s) comprise: (i) a nucleotide sequence that encodes an ER signal sequence, and (ii) a nucleotide sequence that encodes a peptide in frame with the nucleotide sequence that encodes the ER signal sequence, and wherein the recombinant nucleic acid molecule(s) encode a fusion protein comprising the peptide and the ER signal sequence.

2. The method of claim 1, wherein the nucleotide sequence that encodes the peptide is downstream of the nucleotide sequence that encodes ER signal sequence, and wherein the recombinant nucleic acid molecule(s) encode a fusion protein comprising the peptide with an N-terminal ER signal sequence.

3. The method of claim 1, wherein if the T cell, TCR, or TCR-like molecule binds to the engineered target cell, the peptide comprises an epitope of the T cell, TCR, or TCR-like molecule.

4. The method of claim 1, wherein the peptide is an 8-25 amino acid peptide.

5. The method of claim 1, wherein the peptide is an 8-11 amino acid peptide.

6. The method of claim 1, wherein the step of contacting the engineered target cells with the T cells, TCRs, or TCR-like molecules is performed in vitro.

7. The method of claim 1, wherein the step of contacting the engineered target cells with the T cells, TCRs, or TCR-like molecules is performed in vivo.

8. The method of claim 1, wherein the step of performing an assay to determine whether the T cells, TCRs, or TCR-like molecules bind to the engineered target cells is performed in vitro.

9. The method of claim 1, wherein the step of performing an assay to determine whether the T cells, TCRs, or TCR-like molecules bind to the engineered target cells is performed in vivo.

10. The method of claim 1, wherein the assay is performed in vitro and comprises detecting and/or measuring binding by FACS, or by using an affinity column or other solid-phase affinity device, or based on IFN gamma secretion.

11. The method of claim 1, wherein the assay is performed in vivo and comprises detecting and/or measuring an indicator of an immune response.

12. The method of claim 1, further comprising either (a) separating engineered target cells that bind to the T cells, TCRs, or TCR-like molecules from those that don't bind the T cells, TCRs, or TCR-like molecules, or (b) separating engineered target cells that bind to the T cells, TCRs, or TCR-like molecules with higher affinity from those that bind the T cells, TCRs, or TCR-like molecules with lower affinity.

13. The method of claim 12, wherein the separating is performed by FACS or by magnetic bead sorting.

14. The method of claim 1, further comprising isolating and/or amplifying the nucleic acid molecule encoding the peptide from the engineered target cell(s).

15. The method of claim 1, further comprising sequencing the nucleic acid molecule encoding the peptide from the engineered target cell.

16. The method of claim 1, wherein the T cells are naturally occurring T cells.

17. The method of claim 1, wherein the T cells are derived from a human patient treated with Immune Checkpoint Blockade (ICB) therapy.

18. The method of claim 1, wherein the T cells are engineered T cells.

19. The method of claim 18, wherein the engineered T cells are “Chimeric Antigen Receptor T Cells” (“CAR-T cells”).

20. The method of claim 1, wherein the TCRs are naturally occurring TCRs cells.

21. The method of claim 1, wherein the TCR cells are engineered TCRs.

22. The method of claim 1, wherein the TCR-like molecules are selected from the group consisting of: soluble TCRs, TCR mimic antibodies (TCRm), Immune Mobilizing Monoclonal TCRs Against Cancer (“ImmTACs”), and Bi-Specific T Cell Engagers (“BITES”).

23. The method of claim 1, wherein the recombinant nucleic acid molecule(s) further comprise(s) nucleotide sequences both upstream and downstream of the nucleotide sequence that encodes the peptide to enable the nucleotide sequence that encodes the peptide to be isolated, amplified, and/or sequenced.

24. The method of claim 1, wherein the ER signal sequence is an MMTV gp70 ER targeting sequence.

25. The method of claim 1, wherein the nucleotide sequence that encodes the ER signal sequence comprises SEQ ID NO. 1, SEQ ID NO. 5, or SEQ ID NO. 10.

26. The method of claim 1, wherein the nucleotide sequence that encodes the ER signal sequence, and the nucleotide sequence that encodes the peptide, are separated from one another by a spacer comprising one or more amino acids.

27. The method of claim 26, wherein the spacer is a cleavable spacer.

28. The method of claim 26, wherein the spacer can be cleaved by an ER-associated peptidase.

29. The method of claim 1, wherein the nucleotide sequence that encodes the peptide is present in the human genome.

30. The method of claim 1, wherein the nucleotide sequence that encodes the peptide is present in the human exome.

31. The method of claim 1, wherein the peptide is a human proteomic peptide.

32. The method of claim 1, wherein the peptide is a viral peptide.

33. The method of claim 1, wherein the peptide is a microbial peptide.

34. The method of claim 1, wherein the peptide does not exist in nature.

35. The method of claim 1, wherein the peptide is known to be, or predicted to be, an MHC ligand.

36. The method of claim 1, wherein the peptide is an MHC ligand that is unstable in solution or that cannot be made synthetically.

37. The method of claim 1, wherein the peptide is known to be, or predicted to be, an MHC class I ligand.

38. The method of claim 1, wherein the peptide is known to be, or predicted to be, an MHC class II ligand.

39. The method of claim 1, wherein the peptide binds to an MHC molecule with an IC₅₀ of 1 nM to 500 nM.

40. The method of claim 1, wherein the population of engineered target cells comprises a library of nucleic acid molecules that encode at least 100 different peptides.

41. The method of claim 1, wherein the population of engineered target cells comprises a library of nucleic acid molecules that encode at least 500 different peptides.

42. The method of claim 1, wherein the population of engineered target cells comprises a library of nucleic acid molecules that encode at least 1,000 different peptides.

43. The method of claim 1, wherein the population of engineered target cells comprises a library of nucleic acid molecules that encode at least 5,000 different peptides.

44. The method of claim 1, wherein the population of engineered target cells comprises a library of nucleic acid molecules that encode at least 10,000 different peptides

45. The method of claim 1, wherein the nucleic acid molecules are present in a single-copy competent viral vector.

46. The method of claim 1, wherein the engineered target cell is a eukaryotic cell.

47. The method of claim 1, wherein the engineered target cell is a mammalian cell.

48. The method of claim 1, wherein the engineered target cell is a murine cell.

49. The method of claim 1, wherein the engineered target cell is a human cell.

50. The method of claim 1, wherein the engineered target cell is a human T2 cell.

51. The method of claim 1, wherein the engineered target cell expresses MHC I.

52. The method of claim 1, wherein the engineered target cell expresses MHC II.

53. The method of claim 1, wherein the engineered target cell is deficient in one or more components of the cellular antigen presentation machinery.

54. The method of claim 1, wherein the engineered target cell is Tap1-deficient.

55. The method of claim 1, wherein the engineered target cell is Tap2-deficient.

56. A recombinant nucleic acid molecule comprising:
(a) a nucleotide sequence that encodes an ER signal sequence,
(b) a nucleotide sequence that encodes peptide downstream of, and in frame with (a),
(c) a stop codon downstream of (b), and
(d) nucleotide sequences both upstream and downstream of (b) that enable a nucleotide sequence that comprises the nucleotide sequence of (b) to be isolated, amplified, and/or sequenced,
wherein the nucleic acid molecule encodes a fusion protein comprising the peptide with an N-terminal ER signal sequence.

57. The nucleic acid molecule of claim 56, wherein the peptide is an 8-25 amino acid peptide.

58. The nucleic acid molecule of claim 56, wherein the peptide is an 8-11 amino acid peptide.

59. The nucleic acid molecule of claim 56, wherein the ER signal sequence is an MMTV gp70 ER targeting sequence.

60. The nucleic acid molecule of claim 56, wherein the nucleotide sequence that encodes the ER signal sequence comprises SEQ ID NO. 1, SEQ ID NO. 5, or SEQ ID NO. 10.

61. The nucleic acid molecule of claim 56, wherein the nucleotide sequence that encodes the ER signal sequence, and the nucleotide sequence that encodes the peptide, are separated by a spacer comprising one or more amino acids.

62. The nucleic acid molecule of claim 61, wherein the spacer is a cleavable spacer.

63. The nucleic acid molecule of claim 61, wherein the spacer can be cleaved by an ER-associated peptidase.

64. The nucleic acid molecule of claim 56, wherein the nucleotide sequence that encodes the peptide is present in the human genome.

65. The nucleic acid molecule of claim 56, wherein the nucleotide sequence that encodes the peptide is present in the human exome.

66. The nucleic acid molecule of claim 56, wherein the peptide is a human proteomic peptide.

67. The nucleic acid molecule of claim 56, wherein the peptide is a viral peptide.

68. The nucleic acid molecule of claim 56, wherein the peptide is a microbial peptide.

69. The nucleic acid molecule of claim 56, wherein the peptide does not exist in nature.

70. The nucleic acid molecule of claim 56, wherein the peptide is known to be, or predicted to be, an MHC ligand.

71. The nucleic acid molecule of claim 56, wherein the peptide is an MHC ligand that is unstable in solution or that cannot be made synthetically.

72. The nucleic acid molecule of claim 56, wherein the peptide is known to be, or predicted to be, an MHC class I ligand.

73. The nucleic acid molecule of claim 56, wherein the peptide is known to be, or predicted to be, an MHC class II ligand.

74. The nucleic acid molecule of claim 56, wherein the peptide binds to an MEW molecule with an IC₅₀ of 1 nM to 500 nM.

75. The nucleic acid molecule of claim 56, wherein component (d) comprises amplification primer binding sites and/or sequencing primer binding sites that are barcoded for use in a high-throughput sequencing method.

76. The nucleic acid molecule of claim 56, wherein component (d) comprises Illumina signal sequences.

77. The nucleic acid molecule of claim 56, wherein component (d) comprises P5 and P7 Illumina amplification primer binding sites.

78. The nucleic acid molecule of claim 56, wherein component (d) comprises SP1, SP2 and SP3 Illumina sequencing primer binding sites.

79. The nucleic acid molecule of claim 56, wherein component (d) comprises restriction enzyme cleavage sites.

80. The nucleic acid molecule of claim 56, wherein component (d) comprises a pair of identical restriction enzyme cleavage sites.

81. The nucleic acid molecule of claim 56, wherein the nucleic acid molecule is operably linked to a promoter.

82. The nucleic acid molecule of claim 56, wherein the nucleic acid molecule also comprises a selectable marker.

83. The nucleic acid molecule of claim 82, wherein the selectable marker is an antibiotic resistance gene.

84. The nucleic acid molecule of claim 56, wherein the nucleic acid molecule also comprises a detectable marker.

85. The nucleic acid molecule of claim 84, wherein the detectable marker encodes a fluorescent protein.

86. The nucleic acid molecule of claim 85, wherein the fluorescent protein is selected from the group consisting of GFP, RFP, YFP, and CFP.

87. The nucleic acid molecule of claim 56, comprising SEQ ID NO. 1.

88. The nucleic acid molecule of claim 56, comprising SEQ ID NO. 5.

89. The nucleic acid molecule of claim 56, comprising SEQ ID NO. 10.

90. A recombinant nucleic acid molecule comprising SEQ ID NO. 9.

91. A recombinant nucleic acid molecule comprising SEQ ID NO. 10.

92. A recombinant nucleic acid molecule comprising SEQ ID NO. 35.

93. A recombinant nucleic acid molecule comprising SEQ ID NO. 40.

94. A recombinant nucleic acid molecule comprising SEQ ID NO. 41.

95. A vector comprising a nucleic acid molecule according to claim 46.

96. The vector of claim 95, wherein the vector is a single-copy competent viral vector.

97. The vector of claim 95, wherein the vector is a retroviral vector.

98. The vector of claim 95, wherein the vector is a MSCV retroviral vector.

99. The vector of claim 95, comprising SEQ ID NO. 1.

100. The vector of claim 95, comprising SEQ ID NO. 5.

101. The vector of claim 95, comprising SEQ ID NO. 10. SEQ ID NO. 40, or SEQ ID NO. 41.

102. A library comprising multiple nucleic acid molecules according to claim 46, wherein the nucleic acid molecules encode multiple (i.e. two or more) different peptides.

103. A library according to claim 102, comprising nucleic acid molecules that encode at least 100 different peptides.

104. A library according to claim 102, comprising nucleic acid molecules that encode at least 500 different peptides.

105. A library according to claim 102, comprising nucleic acid molecules that encode at least 1,000 different peptides.

106. A library according to claim 102, comprising nucleic acid molecules that encode at least 1,000 different peptides.

107. A library according to claim 102, comprising nucleic acid molecules that encode at least 5,000 different peptides.

108. A library according to claim 102, comprising nucleic acid molecules that encode at least 10,000 different peptides.

109. A library according to claim 102, wherein the nucleic acid molecules are present in a single-copy competent viral vector.

110. A library according to claim 102, comprising a randomly selected group of nucleic acid molecules.

111. A library according to claim 102, comprising nucleic acid molecules that encode a randomly selected group of peptides.

112. A library according to claim 102, comprising nucleic acid molecules that encode peptides known, or predicted, to predicted to bind to an MEW molecule with an IC₅₀ of 1 nM to 500 nM.

113. A library according to claim 102, wherein the MEW molecule is an MHC Class I molecule.

114. A library according to claim 102, wherein the MEW molecule is an MHC Class II molecule.

115. A library according to claim 102, comprising nucleic acid molecules that encode peptides derived from proteins known to be expressed by a given cell type of interest.

116. A library according to claim 102, comprising nucleic acid molecules that encode peptides known, or predicted, to bind to or be cross-reactive with TCRs or TCR like molecules.

117. A library according to claim 102, comprising nucleic acid molecules that encode peptides that are known to, or predicted to, bind to a defined TCR or TCR like molecule.

118. A library according to claim 102, comprising nucleic acid molecules that encode peptides that are known to, or predicted to, be cross-reactive with a defined TCR or TCR like molecule.

119. A virus comprising a nucleic acid molecule according to claim 56.

120. The virus of claim 119, wherein the virus is a retrovirus.

121. The virus of claim 119, wherein the retrovirus is MSCV.

122. A cell comprising a nucleic acid molecule according to claim 56.

123. A cell comprising a vector according to claim 95.

124. A cell comprising a virus according to claim 119.

125. A population of cells comprising a library according to claim 102.

126. The cell according to claim 122, wherein the cell is a eukaryotic cell.

127. The cell according to claim 122, wherein the cell is a mammalian cell.

128. The cell according to claim 122, wherein the cell is a murine cell.

129. The cell according to claim 122, wherein the cell is a human cell.

130. The cell according to claim 122, wherein the cell is a human T2 cell.

131. The cell according to claim 122, wherein the cell expresses MHC I.

132. The cell according to claim 122, wherein the cell expresses MHC II.

133. The cell according to claim 122, wherein the cell is deficient in one or more components of the cellular antigen presentation machinery.

134. The cell according to claim 122, wherein the cell is Tap1-deficient.

135. The cell according to claim 122, wherein the cell is Tap2-deficient.

136. A method of producing a engineered target cell that expresses and on its surface an engineered peptide-MHC (pMHC) complex, the method comprising: culturing a mammalian cell comprising a nucleic acid molecule according to claim 56 under conditions that allow for expression of the fusion protein encoded by said nucleic acid molecule, whereby the peptide within the fusion protein is delivered to the endoplasmic reticulum of the mammalian cell, and associates with MHC molecules in the endoplasmic reticulum of the mammalian cell forming a peptide-MHC (pMHC) complex, and whereby the pMHC complex is presented on the surface of the mammalian cell.

137. The method of claim 136, wherein the mammalian cell is a human cell.

138. The method of claim 136, wherein the mammalian cell is a human T2 cell.

139. The method of claim 136, wherein the mammalian cell expresses MHC class I.

140. The method of claim 136, wherein the mammalian cell expresses MHC class II.

141. The method of claim 136, wherein the mammalian cell is deficient in one or more components of the cellular antigen presentation machinery.

142. The method of claim 136, wherein the mammalian cell is Tap1-deficient.

143. The method of claim 136, wherein the mammalian cell is Tap1-deficient.

144. Use of a composition or method according to any of the preceding claims, to predict or identify targets of T-cells, TCRs, or TCR-like molecules.

145. Use of a composition or method according to any one of claims 1-143, to predict or study the toxicity and/or off-target effects of T-cells, TCRs, or TCR-like molecules.


SEQUENCE LISTING (52 sequences)

SEQ ID NO: 1 - DNA, 294 bp, Artificial Sequence
  Description of Artificial Sequence Synthetic polynucleotide
atgcctaatcatcagtccgggtcacctaccggcagttcagacctgctccttgatggcaagaaacaacgagcccatctggcgctgaggagaaaacggcgacgggaaatgcgcaagattaaccgaaaggtgagaagaatgaatctcgcacccattaaagaaaaaacagcctggcagcacctgcaagctctgatcttcgaggcggaagaagtgttgaagacttctcaaactccacagacctccctcacgctgtttctggcactcttgtctgtactcgggcccccacctgtgagcggg

SEQ ID NO: 2 - protein, 98 aa, Artificial Sequence
  Description of Artificial Sequence Synthetic polypeptide
MPNHQSGSPTGSSDLLLDGKKQRAHLALRRKRRREMRKINRKVRRMNLAPIKEKTAWQHLQALIFEAEEVLKTSQTPQTSLTLFLALLSVLGPPPVSG

SEQ ID NO: 3 - DNA, 391 bp, Artificial Sequence
  Description of Artificial Sequence Synthetic polynucleotide
aattcactgactgactgactgaacatgctcgaggccaccatgcctaatcatcagtccgggtcacctaccggcagttcagacctgctccttgatggcaagaaacaacgagcccatctggcgctgaggagaaaacggcgacgggaaatgcgcaagattaaccgaaaggtgagaagaatgaatctcgcacccattaaagaaaaaacagcctggcagcacctgcaagctctgatcttcgaggcggaagaagtgttgaagacttctcaaactccacagacctccctcacgctgtttctggcactcttgtctgtactcgggcccccacctgtgagcgggaggatgtttcctaacgcgccctacctgtaagaattcgtacaacaactgaccgaatcac

SEQ ID NO: 4 - DNA, 418 bp, Artificial Sequence
  Description of Artificial Sequence Synthetic polynucleotide
aattcactgactgactgactgaacatgctcgaggccaccatggactacaaagacgatgacgacaagcctaatcatcagtccgggtcacctaccggcagttcagacctgctccttgatggcaagaaacaacgagcccatctggcgctgaggagaaaacggcgacgggaaatgcgcaagattaaccgaaaggtgagaagaatgaatctcgcacccattaaagaaaaaacagcctggcagcacctgcaagctctgatcttcgaggcggaagaagtgttgaagacttctcaaactccacagacctccctcacgctgtttctggcactcttgtctgtactcgggcccccacctgtgagcggggctctctatgtggactctttatttttcctttaagaattcgtacaacaactgaccgaatcac

SEQ ID NO: 5 - DNA, 297 bp, Artificial Sequence
  Description of Artificial Sequence Synthetic polynucleotide
atgcctaatcatcagtccgggtcacctaccggttgcagttcagacctgctccttgatggcaagaaacaacgagcccatctggcgctgaggagaaaacggcgacgggaaatgcgcaagattaaccgaaaggtgagaagaatgaatctcgcacccattaaagaaaaaacagcctggcagcacctgcaagctctgatcttcgaggcggaagaagtgttgaagacttctcaaactccacagacctccctcacgctgtttctagcactcttggccgtattggccccgccacctgtgagcggg

SEQ ID NO: 6 - protein, 98 aa, Artificial Sequence
  Description of Artificial Sequence Synthetic polypeptide
MPNHQSGSPTGSSDLLLDGKKQRAHLALRRKRRREMRKINRKVRRMNLAPIKEKTAWQHLQALIFEAEEVLKTSQTPQTSLTLFLALLAVLAPPPVSG

SEQ ID NO: 7 - DNA, 294 bp, Artificial Sequence
  Description of Artificial Sequence Synthetic polynucleotide
atggaaggggccagaagccaactgttgctgaatcacatcttgactcctatgattcttagactgcgagcacaactaattagggcgtggcgcccacatcctaaccaggatacttccacgaaagggaaactggtgcagctctttaaggccaccaagatgccaaaactccatcgaaagaaggagagaccttcattggaaagtgcgggctcttcacaagtgacaccggtagccgcttccaccctcctccgactggtgaaacggcccgagttcaatggccgggcagccgaacaggacctg

SEQ ID NO: 8 - DNA, 294 bp, Artificial Sequence
  Description of Artificial Sequence Synthetic polynucleotide
atgcagctcttgactcctactaagacaaaaaacgaatcagccatggctgccgcgaaggtgtcagcaaagtctcgactcttggcaagaagactgaccgtactcaagttccggccaagcctggacctggaacatagtgtggccattcgcccttccaaacaacgatcccaaatgcagacgcggcttgatctgggcaatcctggccaagtgcagaatccactaggggaagggatcattttttggcacgaagcgcgaaaattgctcaaaagaccccataccctgctggccagggagccg

SEQ ID NO: 9 - DNA, 641 bp, Artificial Sequence
  Description of Artificial Sequence Synthetic polynucleotide
ctgaacatgctcgaggtgactggagttcagacgtgtgctcttccgatcgccaccatgcctaatcatcagtccgggtcacctaccggcagttcagacctgctccttgatggcaagaaacaacgagcccatctggcgctgaggagaaaacggcgacgggaaatgcgcaagattaaccgaaaggtgagaagaatgaatctcgcacccattaaagaaaaaacagcctggcagcacctgcaagctctgatcttcgaggcggaagaagtgttgaagacttctcaaactccacagacctccctcacgctgtttctagcactcttggccgtattggccacatttgcttctgacacaactgtgttcactagcaacctcaaacagacaccatggtgcatctgactcctgaggagaagtctgccgttactgccctgtggggcaaggtgaacgtggatgaagttggtggtgaggccctgggcaggttggtatcaaggttacaagacaggtttaaggagaccaatagaaactgggcatgtggagacagagaagggccaaacaggccaaagatcggaagagcgtcgtgtagggaaagagtgtagatctcggtggtcgccgtatcattgaattcgtacaacaactgaccgaatcac

SEQ ID NO: 10 - DNA, 31 bp, Artificial Sequence
  Description of Artificial Sequence Synthetic oligonucleotide
ggccgtattggccccgccacctgtgagcggg

SEQ ID NO: 11 - DNA, 74 bp, Artificial Sequence
  Description of Artificial Sequence Synthetic oligonucleotide
ggccgtattggccccgccacctgtgagcgggaggatgtttcctaacgcgccctacctgtaaggccaaacaggcc

SEQ ID NO: 12 - DNA, 77 bp, Artificial Sequence
  Description of Artificial Sequence Synthetic oligonucleotide
ggccgtattggccccgccacctgtgagcggggctctctatgtggactctttatttttcctttaaggccaaacaggcc

SEQ ID NO: 13 - DNA, 74 bp, Artificial Sequence
  Description of Artificial Sequence Synthetic oligonucleotide
ggccgtattggccccgccacctgtgagcgggaacctggtgcccatggtggccaccgtgtaaggccaaacaggcc

SEQ ID NO: 14 - DNA, 74 bp, Artificial Sequence
  Description of Artificial Sequence Synthetic oligonucleotide
ggccgtattggccccgccacctgtgagcgggcagctgcagaaccccagctacgacaagtaaggccaaacaggcc

SEQ ID NO: 15 - DNA, 74 bp, Artificial Sequence
  Description of Artificial Sequence Synthetic oligonucleotide
ggccgtattggccccgccacctgtgagcgggggcatcctgggcttcgtgttcaccctgtaaggccaaacaggcc

SEQ ID NO: 16 - DNA, 74 bp, Artificial Sequence
  Description of Artificial Sequence Synthetic oligonucleotide
ggccgtattggccccgccacctgtgagcgggaaccagatgaacctgggcgccaccctgtaaggccaaacaggcc

SEQ ID NO: 17 - protein, 9 aa, Artificial Sequence
  Description of Artificial Sequence Synthetic peptide
LLFGYPVYV

SEQ ID NO: 18 - DNA, 74 bp, Artificial Sequence
  Description of Artificial Sequence Synthetic oligonucleotide
ggccgtattggccccgccacctgtgagcgggctgctttttggttaccctgtttatgtttaaggccaaacaggcc

SEQ ID NO: 19 - DNA, 26 bp, Artificial Sequence
  Description of Artificial Sequence Synthetic oligonucleotide
cgactcactatagggccgtattggcc

SEQ ID NO: 20 - DNA, 23 bp, Artificial Sequence
  Description of Artificial Sequence Synthetic oligonucleotide
agtgatttccggcctgtttggcc

SEQ ID NO: 21 - DNA, 63 bp, Artificial Sequence
  Description of Artificial Sequence Synthetic primer
caagcagaagacggcatacgagatcgtgatgtgactggagttcagacgtgtgctcttccgatc

SEQ ID NO: 22 - DNA, 63 bp, Artificial Sequence
  Description of Artificial Sequence Synthetic primer
caagcagaagacggcatacgagatacatcggtgactggagttcagacgtgtgctcttccgatc

SEQ ID NO: 23 - DNA, 63 bp, Artificial Sequence
  Description of Artificial Sequence Synthetic primer
caagcagaagacggcatacgagatgcctaagtgactggagttcagacgtgtgctcttccgatc

SEQ ID NO: 24 - DNA, 63 bp, Artificial Sequence
  Description of Artificial Sequence Synthetic primer
caagcagaagacggcatacgagattggtcagtgactggagttcagacgtgtgctcttccgatc

SEQ ID NO: 25 - DNA, 63 bp, Artificial Sequence
  Description of Artificial Sequence Synthetic primer
caagcagaagacggcatacgagatcactgtgtgactggagttcagacgtgtgctcttccgatc

SEQ ID NO: 26 - DNA, 63 bp, Artificial Sequence
  Description of Artificial Sequence Synthetic primer
caagcagaagacggcatacgagatattggcgtgactggagttcagacgtgtgctcttccgatc

SEQ ID NO: 27 - DNA, 63 bp, Artificial Sequence
  Description of Artificial Sequence Synthetic primer
caagcagaagacggcatacgagatgatctggtgactggagttcagacgtgtgctcttccgatc

SEQ ID NO: 28 - DNA, 63 bp, Artificial Sequence
  Description of Artificial Sequence Synthetic primer
caagcagaagacggcatacgagattcaagtgtgactggagttcagacgtgtgctcttccgatc

SEQ ID NO: 29 - DNA, 63 bp, Artificial Sequence
  Description of Artificial Sequence Synthetic primer
caagcagaagacggcatacgagatctgatcgtgactggagttcagacgtgtgctcttccgatc

SEQ ID NO: 30 - DNA, 63 bp, Artificial Sequence
  Description of Artificial Sequence Synthetic primer
caagcagaagacggcatacgagataagctagtgactggagttcagacgtgtgctcttccgatc

SEQ ID NO: 31 - DNA, 63 bp, Artificial Sequence
  Description of Artificial Sequence Synthetic primer
caagcagaagacggcatacgagatgtagccgtgactggagttcagacgtgtgctcttccgatc

SEQ ID NO: 32 - DNA, 63 bp, Artificial Sequence
  Description of Artificial Sequence Synthetic primer
caagcagaagacggcatacgagattacaaggtgactggagttcagacgtgtgctcttccgatc

SEQ ID NO: 33 - DNA, 25 bp, Artificial Sequence
  Description of Artificial Sequence Synthetic primer
aatgatacggcgaccaccgagatct

SEQ ID NO: 34 - DNA, 33 bp, Artificial Sequence
  Description of Artificial Sequence Synthetic primer
acgctcttccgatctttggcctgtttggcctta

SEQ ID NO: 35 - DNA, 8486 bp, Artificial Sequence
  Description of Artificial Sequence Synthetic polynucleotide
tgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgtatccaataaaccctcttgcagttgcatccgacttgtggtctcgctgttccttgggagggtctcctctgagtgattgactacccgtcagcgggggtctttcatgggtaacagtttcttgaagttggagaacaacattctgagggtaggagtcgaatattaagtaatcctgactcaattagccactgttttgaatccacatactccaatactcctgaaatagttcattatggacagcgcagaagagctggggagaattaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggcgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggcgattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcggtcgtttcgtgtctgtctctgtctttgtgcgtgtttgtgccggcatctaatgtttgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattagatctctcgactagggataacagggtaattgtttgaatgaggcttcagtactttacagaatcgttgcctgcacatcttggaaacacttgctgggattacttcttcaggttaacccaacagaaggctcgaggtgactggagttcagacgtgtgctcttccgatcgccaccatgcctaatcatcagtccgggtcacctaccggcagttcagacctgctccttgatggcaagaaacaacgagcccatctggcgctgaggagaaaacggcgacgggaaatgcgcaagattaaccgaaaggtgagaagaatgaatctcgcacccattaaagaaaaaacagcctggcagcacctgcaagctctgatcttcgaggcggaagaagtgttgaagacttctcaaactccacagacctccctcacgctgtttctagcactcttggccgtattggccacatttgcttctgacacaactgtgttcactagcaacctcaaacagacaccatggtgcatctgactcctgaggagaagtctgccgttactgccctgtggggcaaggtgaacgtggatgaagttggtggtgaggccctgggcaggttggtatcaaggttacaagacaggtttaaggagaccaatagaaactgggcatgtggagacagagaagggccaaacaggccaaagatcggaagagcgtcgtgtagggaaagagtgtagatctcggtggtcgccgtatcattgaattcaaggggctactttaggagcaattatcttgtttactaaaactgaataccttgctatctctttgatacatttttacaaagctgaattaaaatggtataaattaaatcacttttttcaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatcatccaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgaattaattaagaattatcaagcttatcgataccgtcgacctgcagccaagcttatcgataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccc

SEQ ID NO: 36 - protein, 9 aa, Artificial Sequence
  Description of Artificial Sequence Synthetic peptide
RMFPNAPYL

SEQ ID NO: 37 - protein, 10 aa, Artificial Sequence
  Description of Artificial Sequence Synthetic peptide
ALYVDSLFFL

SEQ ID NO: 38 - DNA, 25 bp, Artificial Sequence
  Description of Artificial Sequence Synthetic primer
aattcactgactgactgactgaaca

SEQ ID NO: 39 - DNA, 23 bp, Artificial Sequence
  Description of Artificial Sequence Synthetic primer
gtgattcggtcagttgttgtacg

SEQ ID NO: 40 - DNA, 31 bp, Artificial Sequence; identical to SEQ ID NO: 10
  Description of Artificial Sequence Synthetic oligonucleotide

SEQ ID NO: 41 - DNA, 16 bp, Artificial Sequence
  Description of Artificial Sequence Synthetic oligonucleotide
taaggccaaacaggcc

SEQ ID NO: 42 - protein, 9 aa, Artificial Sequence
  Description of Artificial Sequence Synthetic peptide
QLQNPSYDK

SEQ ID NO: 43 - protein, 9 aa, Artificial Sequence
  Description of Artificial Sequence Synthetic peptide
GILGFVFTL

SEQ ID NO: 44 - protein, 9 aa, Artificial Sequence
  Description of Artificial Sequence Synthetic peptide
NQMNLGATL

SEQ ID NO: 45 - protein, 15 aa, Artificial Sequence
  Description of Artificial Sequence Synthetic peptide
GLNDIFEAQKIEWHE

SEQ ID NO: 46 - DNA, 45 bp, Artificial Sequence
  Description of Artificial Sequence Synthetic oligonucleotide
ggcctgaacgatatttttgaagcgcagaaaattgaatggcatgaa

SEQ ID NO: 47 - protein, 9 aa, Artificial Sequence
  Description of Artificial Sequence Synthetic peptide
NLVPMVATV

SEQ ID NO: 48 - DNA, 43 bp, Artificial Sequence
  Description of Artificial Sequence Synthetic oligonucleotide
gcagaaaattgaatggcatgaataagcttgaattccgatccgg

SEQ ID NO: 49 - DNA, 41 bp, Artificial Sequence
  Description of Artificial Sequence Synthetic oligonucleotide
gcttcaaaaatatcgttcaggccgtctgctctaccccaggc

SEQ ID NO: 50 - protein, 18 aa, Artificial Sequence
  Description of Artificial Sequence Synthetic peptide
  MOD_RES (9)..(9): Ser or Ala
  MOD_RES (12)..(12): Gly or Ala
LTLFLALLXVLXPPPVSG

SEQ ID NO: 51 - protein, 18 aa, Artificial Sequence
  Description of Artificial Sequence Synthetic peptide
LTLFLALLSVLGPPPVSG

SEQ ID NO: 52 - protein, 18 aa, Artificial Sequence
  Description of Artificial Sequence Synthetic peptide
LTLFLALLAVLAPPPVSG